SEQUENCE LISTING (55 sequences)

SEQ ID NO: 1 - protein, 7 aa, Human immunodeficiency virus
KRRQRRR

SEQ ID NO: 2 - protein, 16 aa, Artificial Sequence
  Description of Artificial Sequence Synthetic peptide
RQIKIWFQNRRMKWKK

SEQ ID NO: 3 - protein, 34 aa, Human herpesvirus
DAATATRGRSAASRPTERPRAPARSASRPRRPVD

SEQ ID NO: 4 - protein, 16 aa, Artificial Sequence
  Description of Artificial Sequence Synthetic peptide
AAVALLPAVLLALLAP

SEQ ID NO: 5 - protein, 16 aa, Artificial Sequence
  Description of Artificial Sequence Synthetic peptide
AAVLLPVLLPVLLAAP

SEQ ID NO: 6 - protein, 15 aa, Homo sapiens
VTVLALGALAGVGVG

SEQ ID NO: 7 - protein, 17 aa, Human immunodeficiency virus
GALFLGWLGAAGSTMGA

SEQ ID NO: 8 - protein, 17 aa, Caiman crocodilus
MGLGLHLLVLAAALQGA

SEQ ID NO: 9 - protein, 24 aa, Homo sapiens
LGTYTQDFNKFHTFPQTAIGVGAP

SEQ ID NO: 10 - protein, 26 aa, Artificial Sequence
  Description of Artificial Sequence Synthetic peptide
GWTLNSAGYLLKINLKALAALAKKIL

SEQ ID NO: 11 - protein, 16 aa, Artificial Sequence
  Description of Artificial Sequence Synthetic peptide
TPPKKKRKVEDPKKKK

SEQ ID NO: 12 - protein, 7 aa, Artificial Sequence
  Description of Artificial Sequence Synthetic peptide
RRRRRRR

SEQ ID NO: 13 - protein, 18 aa, Artificial Sequence
  Description of Artificial Sequence Synthetic peptide
KLALKLALKALKAALKLA

SEQ ID NO: 14 - protein, 16 aa, Influenza virus
GLFGAIAGFIENGWEG

SEQ ID NO: 15 - protein, 16 aa, Human parainfluenza virus
FFGAVIGTIALGVATA

SEQ ID NO: 16 - protein, 16 aa, Respiratory syncytial virus
FLGFLLGVGSAIASGV

SEQ ID NO: 17 - protein, 16 aa, Human immunodeficiency virus
GVFVLGFLGFLATAGS

SEQ ID NO: 18 - protein, 16 aa, Ebola virus
GAAIGLAWIPYFGPAA

SEQ ID NO: 19 - protein, 56 aa, Artificial Sequence
  Description of Artificial Sequence Synthetic polypeptide
ACTCPYCKDSEGRGSGDPGKKKQHICHIQGCGKVYGKTSHLRAHLRWHTGERPFMC

SEQ ID NO: 20 - protein, 54 aa, Artificial Sequence
  Description of Artificial Sequence Synthetic polypeptide
ACTCPNCKDGEKRSGEQGKKKHVCHIPDCGKTFRKTSLLRAHVRLHTGERPFVC

SEQ ID NO: 21 - protein, 55 aa, Artificial Sequence
  Description of Artificial Sequence Synthetic polypeptide
ACTCPNCKEGGGRGTNLGKKKQHICHIPGCGKVYGKTSHLRAHLRWHSGERPFVC

SEQ ID NO: 22 - protein, 56 aa, Artificial Sequence
  Description of Artificial Sequence Synthetic polypeptide
ACSCPNCREGEGRGSNEPGKKKQHICHIEGCGKVYGKTSHLRAHLRWHTGERPFIC

SEQ ID NO: 23 - protein, 60 aa, Drosophila melanogaster
RCTCPNCTNEMSGLPPIVGPDERGRKQHICHIPGCERLYGKASHLKTHLRWHTGERPFLC

SEQ ID NO: 24 - protein, 58 aa, Drosophila melanogaster
TCDCPNCQEAERLGPAGVHLRKKNIHSCHIPGCGKVYGKTSHLKAHLRWHTGERPFVC

SEQ ID NO: 25 - protein, 53 aa, Caenorhabditis elegans
RCTCPNCKAIKHGDRGSQHTHLCSVPGCGKTYKKTSHLRAHLRKHTGDRPFVC

SEQ ID NO: 26 - protein, 56 aa, Artificial Sequence
  Description of Artificial Sequence Synthetic polypeptide
PQISLKKKIFFFIFSNFRGDGKSRIHICHLCNKTYGKTSHLRAHLRGHAGNKPFAC

SEQ ID NO: 27 - protein, 31 aa, Artificial Sequence
  Description of Artificial Sequence Synthetic peptide
WWETWKPFQCRICMRNFSTRQARRNHRRRHR

SEQ ID NO: 28 - protein, 16 aa, Artificial Sequence
  Description of Artificial Sequence Synthetic peptide
GKINLKALAALAKKIL

SEQ ID NO: 29 - protein, 16 aa, Artificial Sequence
  Description of Artificial Sequence Synthetic peptide
RVIRVWFQNKRCKDKK

SEQ ID NO: 30 - protein, 39 aa, Artificial Sequence
  Description of Artificial Sequence Synthetic peptide
GRKKRRQRRRPPQGRKKRRQRRRPPQGRKKRRQRRRPPQ

SEQ ID NO: 31 - protein, 22 aa, Artificial Sequence
  Description of Artificial Sequence Synthetic peptide
GEQIAQLIAGYIDIILKKKKSK

SEQ ID NO: 32 - DNA, 21 bp, Artificial Sequence
  Description of Combined DNA/RNA Molecule Synthetic oligonucleotide
  Description of Artificial Sequence Synthetic oligonucleotide
ccgucagccgauuugcuautt

SEQ ID NO: 33 - DNA, 21 bp, Artificial Sequence
  Description of Combined DNA/RNA Molecule Synthetic oligonucleotide
  Description of Artificial Sequence Synthetic oligonucleotide
auagcaaaucggcugacggtt

SEQ ID NO: 34 - DNA, 21 bp, Artificial Sequence
  Description of Combined DNA/RNA Molecule Synthetic oligonucleotide
  Description of Artificial Sequence Synthetic oligonucleotide
gggucggaacccaagcuuatt

SEQ ID NO: 35 - DNA, 21 bp, Artificial Sequence
  Description of Combined DNA/RNA Molecule Synthetic oligonucleotide
  Description of Artificial Sequence Synthetic oligonucleotide
uaagcuuggguuccgacccta

SEQ ID NO: 36 - DNA, 21 bp, Artificial Sequence
  Description of Combined DNA/RNA Molecule Synthetic oligonucleotide
  Description of Artificial Sequence Synthetic oligonucleotide
cuacacaaaucagcgauuutt

SEQ ID NO: 37 - DNA, 21 bp, Artificial Sequence
  Description of Combined DNA/RNA Molecule Synthetic oligonucleotide
  Description of Artificial Sequence Synthetic oligonucleotide
aaaucgcugauuuguguagtc

SEQ ID NO: 38 - DNA, 21 bp, Artificial Sequence
  Description of Combined DNA/RNA Molecule Synthetic oligonucleotide
  Description of Artificial Sequence Synthetic oligonucleotide
cgggacucuagcauacuuatt

SEQ ID NO: 39 - DNA, 21 bp, Artificial Sequence
  Description of Combined DNA/RNA Molecule Synthetic oligonucleotide
  Description of Artificial Sequence Synthetic oligonucleotide
uaaguaugcuagagucccgtt

SEQ ID NO: 40 - DNA, 21 bp, Artificial Sequence
  Description of Combined DNA/RNA Molecule Synthetic oligonucleotide
  Description of Artificial Sequence Synthetic oligonucleotide
acugacagccagacagcgatt

SEQ ID NO: 41 - DNA, 21 bp, Artificial Sequence
  Description of Combined DNA/RNA Molecule Synthetic oligonucleotide
  Description of Artificial Sequence Synthetic oligonucleotide
ucgcugucuggcugucagutt

SEQ ID NO: 42 - DNA, 21 bp, Artificial Sequence
  Description of Combined DNA/RNA Molecule Synthetic oligonucleotide
  Description of Artificial Sequence Synthetic oligonucleotide
agacagcgaccaaaagaautt

SEQ ID NO: 43 - DNA, 21 bp, Artificial Sequence
  Description of Combined DNA/RNA Molecule Synthetic oligonucleotide
  Description of Artificial Sequence Synthetic oligonucleotide
auucuuuuggucgcugucutt

SEQ ID NO: 44 - DNA, 21 bp, Artificial Sequence
  Description of Combined DNA/RNA Molecule Synthetic oligonucleotide
  Description of Artificial Sequence Synthetic oligonucleotide
augaagaucuguuccaccatt

SEQ ID NO: 45 - DNA, 21 bp, Artificial Sequence
  Description of Combined DNA/RNA Molecule Synthetic oligonucleotide
  Description of Artificial Sequence Synthetic oligonucleotide
ugguggaacagaucuucautt

SEQ ID NO: 46 - DNA, 21 bp, Artificial Sequence
  Description of Combined DNA/RNA Molecule Synthetic oligonucleotide
  Description of Artificial Sequence Synthetic oligonucleotide
gaucuguuccaccauugaatt

SEQ ID NO: 47 - DNA, 21 bp, Artificial Sequence
  Description of Combined DNA/RNA Molecule Synthetic oligonucleotide
  Description of Artificial Sequence Synthetic oligonucleotide
uucaaugguggaacagauctt

SEQ ID NO: 48 - DNA, 21 bp, Artificial Sequence
  Description of Combined DNA/RNA Molecule Synthetic oligonucleotide
  Description of Artificial Sequence Synthetic oligonucleotide
gcaauugaggagugccugatt

SEQ ID NO: 49 - DNA, 21 bp, Artificial Sequence
  Description of Combined DNA/RNA Molecule Synthetic oligonucleotide
  Description of Artificial Sequence Synthetic oligonucleotide
ucaggcacuccucaauugctt

SEQ ID NO: 50 - DNA, 21 bp, Artificial Sequence
  Description of Combined DNA/RNA Molecule Synthetic oligonucleotide
  Description of Artificial Sequence Synthetic oligonucleotide
uugaggagugccugauuaatt

SEQ ID NO: 51 - DNA, 21 bp, Artificial Sequence
  Description of Combined DNA/RNA Molecule Synthetic oligonucleotide
  Description of Artificial Sequence Synthetic oligonucleotide
uuaaucaggcacuccucaatt

SEQ ID NO: 52 - DNA, 21 bp, Artificial Sequence
  Description of Combined DNA/RNA Molecule Synthetic oligonucleotide
  Description of Artificial Sequence Synthetic oligonucleotide
ggaucuuauuucuucggagtt

SEQ ID NO: 53 - DNA, 21 bp, Artificial Sequence
  Description of Combined DNA/RNA Molecule Synthetic oligonucleotide
  Description of Artificial Sequence Synthetic oligonucleotide
cuccgaagaaauaagaucctt

SEQ ID NO: 54 - protein, 28 aa, Artificial Sequence
  Description of Artificial Sequence Synthetic peptide
KETWWETWWTEWSQPGRKKRRQRRRPPQ

SEQ ID NO: 55 - protein, 23 aa, Artificial Sequence
  Description of Artificial Sequence Synthetic peptide
  MOD_RES (2)..(5): Variable amino acid and this region may encompass 2 or 4 residues
  MOD_RES (7)..(18): Variable amino acid
  MOD_RES (20)..(22): Variable amino acid
CXXXXCXXXXXXXXXXXXHXXXH

(19) United States
(12) Patent Application Publication
(10) Pub. No.: US 2007/0293449 A1
(43) Pub. Date: Dec. 20, 2007

(54) COMPOSITIONS AND METHODS FOR DELIVERY OF DOUBLE-STRANDED RNA

(75) Inventors: Kunyuan Cui, Bothell, WA (US); Roger C. Adami, Snohomish, WA (US); Dong Liang, Everett, WA (US)

Correspondence Address: NASTECH PHARMACEUTICAL COMPANY INC, 3830 MONTE VILLA PARKWAY, BOTHELL, WA, 98021-7266, US

(73) Assignee: NASTECH PHARMACEUTICAL COMPANY INC., Bothell, WA (US)

(21) Appl. No.: 11/766,038

(22) Filed: Jun. 20, 2007

Related U.S. Application Data
(60) Provisional application No. 60/896,783, filed on Mar. 23, 2007.
(60) Provisional application No. 60/805,327, filed on Jun. 20, 2006.

(51) Int. Cl. A61K 48/00; C12N 15/88

(52) U.S. Cl. 514/44; 435/458

(57) ABSTRACT

Pharmaceutical compositions and methods of use of a composition containing a double-stranded RNA (dsRNA), cationic lipids, non-cationic lipids, and lipophilic delivery-enhancing compounds.

This application claims the benefit under 35 U.S.C. §119(e) of U.S. Provisional Application Nos. 60/805,327, filed Jun. 20, 2006, and 60/896,783, filed Mar. 23, 2007, which are hereby incorporated by reference in entirety.

### BACKGROUND

RNA interference (RNAi) refers to methods of sequence-specific post-transcriptional gene silencing which is mediated by a double-stranded RNA (dsRNA) called a short interfering RNA (siRNA). See Fire, et al, Nature 391:806, 1998, and Hamilton, et al., Science 286:950-951, 1999. RNAi is shared by diverse flora and phyla and is believed to be an evolutionarily-conserved cellular defense mechanism against the expression of foreign genes. See Fire, et al., Trends Genet. 15:358, 1999.

RNAi is therefore an endogenous mechanism that uses small noncoding RNAs to silence gene expression. When an siRNA is introduced into a cell, it binds to the endogenous RNAi machinery to alter the level of mRNA containing complementary sequences with high specificity. Various disease-related genes, cell types or tissues can potentially be targeted. Utilizing RNAi holds great promise for therapeutic applications, however, introducing siRNAs into cells in vivo is a major obstacle.

The mechanism of RNAi, although not yet fully characterized, is through cleavage of a target mRNA. The RNAi response involves an endonuclease complex known as the RNA-induced silencing complex (RISC), which mediates cleavage of a single-stranded RNA complementary to the antisense strand of the siRNA duplex. Cleavage of the target RNA takes place in the middle of the region complementary to the antisense strand of the siRNA duplex (Elbashir, et al., Genes Dev. 15:188, 2001).

One way to carry out RNAi is to introduce or express a siRNA in cells. Another way is to make use of an endogenous ribonuclease III enzyme called dicer. One activity of dicer is to process a long dsRNA into siRNAs. See Hamilton, et al., Science 286:950-951, 1999; Berstein, et al., Nature 409:363, 2001. A siRNA derived from dicer is typically about 21-23 nucleotides in overall length with about 19 base pairs duplexed. See Hamilton, et al., supra; Elbashir, et al., Genes Dev. 15:188, 2001. In essence, a long dsRNA can be introduced in a cell as a precursor of a siRNA.

The development of RNAi therapy, among others, has created a need for effective means of introducing active nucleic acid-based agents into cells. In general, nucleic acids are stable for only very limited times in cells or plasma. However, nucleic acid-based agents can be stabilized in compositions and formulations which may be dispersed for cellular delivery.

What is needed are compounds, compositions and formulations for intracellular and in vivo delivery of a nucleic acid agent for use, ultimately, as a therapeutic, which maintain cytoprotection and relatively low toxicity. Furthermore, there is a need for compositions and methods to deliver double-stranded RNA to cells. Moreover, there is a need for compositions and methods for delivery of interfering RNAs to selected cells, tissues, or compartments to modulate gene expression in a manner that will alter a phenotype or disease state.

### BRIEF SUMMARY OF THE DISCLOSURE

This invention satisfies these needs and fulfills additional objects and advantages by providing a range of novel compositions, formulations and methods that employ a short interfering nucleic acid (siNA), or a precursor thereof, in combination with various components including lipids, peptides, and polymers.

In some embodiments, this invention includes pharmaceutical compositions containing a double-stranded RNA (dsRNA), one or more cationic lipids, one or more non-cationic lipids, and one or more lipophilic delivery-enhancing compounds.

In some aspects, this invention includes pharmaceutical compositions wherein a lipophilic delivery-enhancing compound is an omega-3 fatty acid.

In some embodiments, this invention includes pharmaceutical compositions wherein a lipophilic delivery-enhancing compound is a Vitamin E compound, a tocol, a tocotrienol, or a tocopherol.

In some embodiments, this invention includes pharmaceutical compositions wherein the dsRNA is an siRNA or an shRNA, the structure of which may include a 3′ overhang. In some embodiments, the dsRNA has a 3′ overhang which includes a deoxythymidine (dT). A shRNA can include a hairpin loop structure.

In some embodiments, this invention includes pharmaceutical compositions containing a double-stranded RNA (dsRNA), one or more cationic lipids, one or more non-cationic lipids, and one or more peptides.

Compositions of this invention may decrease expression of an influenza A virus gene by at least about 25% in a mammalian cell.

In some embodiments, this disclosure includes methods for delivering a dsRNA to a cell and/or inhibiting expression of a gene in a cell. This invention also includes methods for inhibiting expression of a gene in a mammal and methods for treating diseases such as influenza in a mammal.

### DETAILED DESCRIPTION OF THE DISCLOSURE

This disclosure provides a range of compounds, compositions, formulations and methods which include an interfering ribonucleic acid, or a precursor thereof, in combination with various components including lipids, peptides, and delivery components.

This invention relates generally to the fields of gene-specific inhibition of gene expression in mammals and delivery of double-stranded ribonucleic acids. More particularly, this invention relates to compounds, compositions and methods of using double-stranded ribonucleic acids with lipids, peptides, and lipophilic delivery-enhancing compounds.

In some embodiments, this disclosure provides novel compositions and methods to facilitate the delivery of RNAi-inducing agents such as short interfering siRNAs (siRNAs), short hairpin RNAs (shRNAs), and/or RNAi-inducing vectors to cells, tissues, and organs in living mammals, e.g., humans, or to provide medicaments thereof.

In some embodiments, this invention provides compositions containing one or more RNAi-inducing agents which are targeted to one or more target transcripts, along with one or more delivery components. Examples of delivery components include neutral lipids, anionic lipids, cationic lipids, peptides, liposomes, surfactants, and polymers.

The compositions and formulations of this disclosure may be used for delivery of RNAi-inducing entities such as siRNA, shRNA, or RNAi-inducing vectors to cells in intact mammalian subjects, or may also be used for delivery of these agents to cells in culture.

This invention also provides methods for the delivery of one or more RNAi-inducing entities to organs and tissues within the body of a mammal, for example, a human. In some embodiments, compositions containing an RNAi-inducing entity and one or more delivery components are introduced by various routes to be transported within the body and taken up by cells in one or more organs or tissues, where it can modulate expression of a target transcript.

This disclosure provides pharmaceutically acceptable nucleic acid compositions useful for therapeutic delivery of nucleic acids and gene-silencing RNAs. In particular, this invention provides compositions and methods for in vitro and in vivo delivery of RNAs decreasing, downregulating, or silencing the translation of a target nucleic acid sequence or expression of a gene. These compositions and methods may be used for prevention and/or treatment of diseases in a mammal. In these methods, a ribonucleic acid molecule such as an siRNA or shRNA is contacted with delivery components to formulate a nucleic acid composition which can be administered to cells or subjects, e.g., mammals or humans. In some embodiments, this invention provides a method for delivering an siRNA or shRNA intracellularly by contacting a nucleic acid composition with a cell.

In some embodiments, nucleic acid compositions of this invention may contain an RNA, a lipid, a peptide, and carrier or excipient components.

In some embodiments, nucleic acid compositions of this invention may contain an RNA, a cationic lipid, a non-cationic lipid, and one or more delivery-enhancing lipophilic components. Delivery-enhancing lipophilic components may include omega-3 oils and lipid-soluble compounds such as tocopherols and Vitamin E.

In exemplary embodiments, this disclosure includes compositions containing an interfering nucleic acid molecule, such as short interfering nucleic acid (siNA), a short interfering RNA (siRNA), a double-stranded RNA (dsRNA), or a short hairpin RNA (shRNA), admixed or complexed with one or more cationic lipids, one or more non-cationic lipids, or a combination of one or more cationic lipids and one or more non-cationic lipids to form a composition that enhances intracellular delivery of the nucleic acid molecule.

Within novel compositions of this invention, an interfering RNA may be admixed or complexed with lipids and/or peptides to form a composition that enhances intracellular delivery of the siNA as compared to contacting target cells with a naked siNA.

In some embodiments, a composition of this invention may contain one or more cationic lipids, where the cationic lipids can be present from about 0.5% to about 70% (mol %) of the total amount of lipid and delivery-enhancing lipophilic components including any polymeric component. In some embodiments, a composition of this invention may contain one or more cationic lipids, where the cationic lipids can be present from about 0.5% to about 30%. In some embodiments, a composition of this invention may contain one or more cationic lipids, where the cationic lipids can be present from about 2% to about 15%.

In some embodiments, a composition of this invention may contain one or more non-cationic lipids, where the non-cationic lipids can be present from about 2% to about 95% (mol %) of the total amount of lipid and delivery-enhancing lipophilic components including any polymeric component. In some embodiments, a composition of this invention may contain one or more non-cationic lipids, where the non-cationic lipids can be present from about 2% to about 85%, from about 2% to about 75%, or from about 10% to about 70%. In some embodiments, a composition of this invention may contain one or more non-cationic lipids, where the non-cationic lipids can be present from about 10% to about 50%.

In some embodiments, a composition of this invention may contain one or more polymeric compounds or polymer-lipid conjugates, where the polymeric compounds or polymer-lipid conjugates can be present from about 0.5% to about 20% (mol %) of the total amount of lipid and delivery-enhancing lipophilic components including any polymeric component. In some embodiments, a composition of this invention may contain one or more polymeric compounds, where the polymeric compounds can be present from about 0.5% to about 10% of the composition. In some embodiments, a composition of this invention may contain one or more polymeric compounds, where the polymeric compounds can be present from about 1% to about 5% of the composition.

Lipids for RNA Delivery and Administration

Lipids for delivery and administration of RNA components include cationic lipids and non-cationic lipids. Cationic lipids may be monocationic or polycationic. Some non-cationic lipids include neutral lipids and lipids having approximately zero net charge at a particular pH, for example, a zwitterionic lipid. Non-cationic lipids also include anionic lipids.

In some embodiments, the composition is a mixture or complex of an siNA with a cationic lipid.

Examples of cationic lipids include N-[1-(2,3-dioleoyloxy)propyl]-N,N,N-trimethylammonium chloride (DOTMA); 1,2-bis(oleoyloxy)-3-3-(trimethylammonium)propane (DOTAP), 1,2-bis(dimyrstoyloxy)-3-3-(trimethylammonia)propane (DMTAP); 1,2-dimyristyloxypropyl-3-dimethylhydroxyethylammonium bromide (DMRIE); dimethyldioctadecylammonium bromide (DDAB); 3-(N-(N′,N′-dimethylaminoethane)carbamoyl)cholesterol (DC-Chol); 3β-[N′,N′-diguanidinoethyl-aminoethane)carbamoyl cholesterol (BGTC); 2-(2-(3-(bis(3-aminopropyl)amino)propylamino)acetamido)-N,N-ditetradecylacetamide (RPR209120); pharmaceutically acceptable salts thereof, and mixtures thereof.

Examples of cationic lipids include 1,2-dialkenoyl-sn-glycero-3-ethylphosphocholines (EPCs), such as 1,2-dioleoyl-sn-glycero-3-ethylphosphocholine, 1,2-distearoyl-sn-glycero-3-ethylphosphocholine, 1,2-dipalmitoyl-sn-glycero-3-ethylphosphocholine, pharmaceutically acceptable salts thereof, and mixtures thereof.

Examples of polycationic lipids include tetramethyltetrapalmitoyl spermine (TMTPS), tetramethyltetraoleyl spermine (TMTOS), tetramethlytetralauryl spermine (TMTLS), tetramethyltetramyristyl spermine (TMTMS), tetramethyldioleyl spermine (TMDOS), pharmaceutically acceptable salts thereof, and mixtures thereof.

Examples of polycationic lipids include 2,5-bis(3-aminopropylamino)-N-(2-(dioctadecylamino)-2-oxoethyl) pentanamide (DOGS); 2,5-bis(3-aminopropylamino)-N-(2-(di(Z)-octadeca-9-dienylamino)-2-oxoethyl) pentanamide (DOGS-9-en); 2,5-bis(3-aminopropylamino)-N-(2-(di(9Z,12Z)-octadeca-9,12-dienylamino)-2-oxoethyl) pentanamide (DLinGS); 3-beta-(N⁴—(N¹,N⁸-dicarbobenzoxyspermidine)carbamoyl)cholesterol (GL-67); (9Z,9′Z)-2-(2,5-bis(3-aminopropylamino)pentanamido)propane-1,3-diyl-dioctadec-9-enoate (DOSPER); 2,3-dioleyloxy-N-[2(sperminecarboxamido)ethyl]-N,N-dimethyl-1-propanaminium trifluoro-acetate (DOSPA); pharmaceutically acceptable salts thereof, and mixtures thereof.

Examples of cationic lipids include those shown in Table 1.

TABLE 1
Examples of Cationic Lipids

| Compound Name | FA Chains | M.W. | CAS registry # |
|---|---|---|---|
| DS404-28 BGTC | Cholesterol | 642.96 | 182056-06-0 |
| DOSPER | C18:1 | 848.34 | 178532-92-8 |
| GL-67 | Cholesterol | 615.00 | 179075-30-0 |
| RPR209120 | Myristoyl C14 | 695.16 | 433292-13-8 |
| DOGS | C18:0 | 807.37 | 12050-77-7 |
| DOGS (9-en) | C18:1 | 803.34 | |
| DLinGS | C18:2 | 799.31 | |
| DOTMA | C18:1 | 712.57 | 104162-48-3 |

Examples of cationic lipids are described in U.S. Pat. Nos. 4,897,355; 5,279,833; 6,733,777; 6,376,248; 5,736,392; 5,686,958; 5,334,761; 5,459,127; 2005/0064595; 5,208,036; 5,264,618; 5,279,833; 5,283,185; 5,753,613; and 5,785,992.

The chemical structure of 3β-[N—(N′,N′-Dimethylaminoethane)-carbamoyl] Cholesterol Hydrochloride (DC-Cholesterol HCl) is as follows:

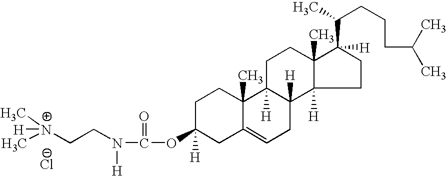

The chemical structure of 1,2-Dioleoyl-3-Trimethylammonium-Propane (chloride salt; DOTAP) is as follows:

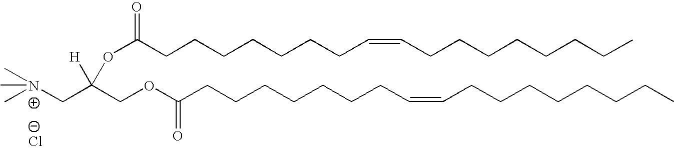

The chemical structure of 1,2-Dioleoyl-sn-Glycero-3-Ethylphosphocholine (EPC) (chloride salt) is as follows:

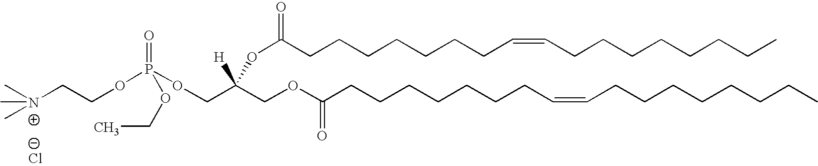

The chemical structure of 1,2-Distearoyl-sn-Glycero-3-Ethylphosphocholine (chloride salt) is as follows:

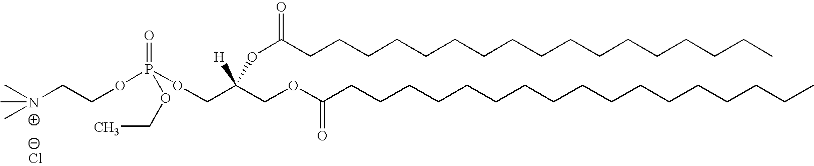

The chemical structure of 1,2-Dipalmitoyl-sn-Glycero-3-Ethylphosphocholine (Chloride Salt) is as follows:

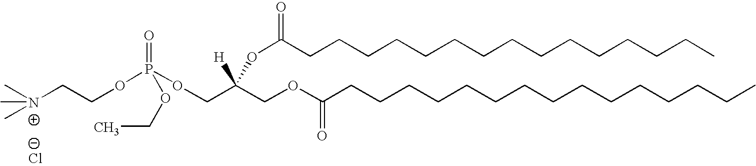

The chemical structure of 1,2-Dimyristoyl-3-Trimethylammonium-Propane (chloride salt) is as follows:

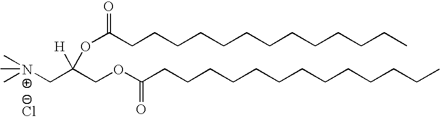

The chemical structure of 1,2-Distearoyl-3-Trimethylammonium-Propane (chloride salt) is as follows:

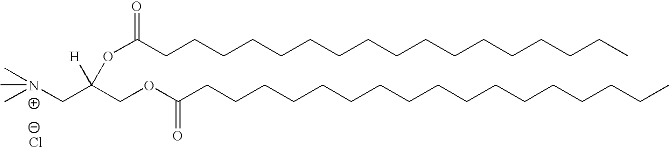

The chemical structure of Dimethyldioctadecylammonium (bromide salt; DDAB) is as follows:

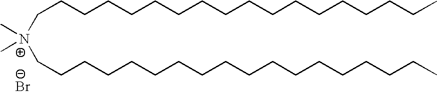

In some embodiments, the composition is a mixture or complex of an RNA component with a non-cationic lipid. In some embodiments, the composition is a mixture or complex of one or more RNA components with one or more cationic lipids and one or more non-cationic lipids. Non-cationic lipids include neutral, zwitterionic, and anionic lipids.

Examples of non-cationic lipids include 1,2-Dilauroyl-sn-glycerol (DLG); 1,2-Dimyristoyl-sn-glycerol (DMG); 1,2-Dipalmitoyl-sn-glycerol (DPG); 1,2-Distearoyl-sn-glycerol (DSG); 1,2-Dilauroyl-sn-glycero-3-phosphatidic acid (sodium salt; DLPA); 1,2-Dimyristoyl-sn-glycero-3-phosphatidic acid (sodium salt; DMPA); 1,2-Dipalmitoyl-sn-glycero-3-phosphatidic acid (sodium salt; DPPA); 1,2-Distearoyl-sn-glycero-3-phosphatidic acid (sodium salt; DSPA); 1,2-Diarachidoyl-sn-glycero-3-phosphocholine (DAPC); 1,2-Dilauroyl-sn-glycero-3-phosphocholine (DLPC); 1,2-Dimyristoyl-sn-glycero-3-phosphocholine (DMPC); 1,2-Dipalmitoyl-sn-glycero-0-ethyl-3-phosphocholine (chloride or triflate; DPePC); 1,2-Dipalmitoyl-sn-glycero-3-phosphocholine (DPPC); 1,2-Distearoyl-sn-glycero-3-phosphocholine (DSPC); 1,2-Dilauroyl-sn-glycero-3-phosphoethanolamine (DLPE); 1,2-Dimyristoyl-sn-glycero-3-phosphoethanolamine (DMPE); 1,2-Dipalmitoyl-sn-glycero-3-phosphoethanolamine (DPPE); 1,2-Distearoyl-sn-glycero-3-phosphoethanolamine (DSPE); 1,2-Dilauroyl-sn-glycero-3-phosphoglycerol (sodium salt; DLPG); 1,2-Dimyristoyl-sn-glycero-3-phosphoglycerol (sodium salt; DMPG); 1,2-Dimyristoyl-sn-glycero-3-phospho-sn-1-glycerol (ammonium salt; DMP-sn-1-G); 1,2-Dipalmitoyl-sn-glycero-3-phosphoglycerol (sodium salt; DPPG); 1,2-Distearoyl-sn-glycero-3-phosphoglycero (sodium salt; DSPG); 1,2-Distearoyl-sn-glycero-3-phospho-sn-1-glycerol (sodium salt; DSP-sn-1-G); 1,2-Dipalmitoyl-sn-glycero-3-phospho-L-serine (sodium salt; DPPS); 1-Palmitoyl-2-linoleoyl-sn-glycero-3-phosphocholine (PLinoPC); 1-Palmitoyl-2-oleoyl-sn-glycero-3-phosphocholine (POPC); 1-Palmitoyl-2-oleoyl-sn-glycero-3-phosphoglycerol (sodium salt; POPG); 1-Palmitoyl-2-oleoyl-sn-glycero-3-phosphoglycerol (sodium salt; POPG); 1-Palmitoyl-2-oleoyl-sn-glycero-3-phosphoglycerol (ammonium salt; POPG); 1-Palmitoyl-2-lyso-sn-glycero-3-phosphocholine (P-lyso-PC); 1-Stearoyl-2-lyso-sn-glycero-3-phosphocholine (S-lyso-PC); and mixtures thereof.

Examples of non-cationic lipids include polymeric compounds and polymer-lipid conjugates, such as pegylated lipids, including polyethyleneglycols, N-(Carbonyl-methoxypolyethyleneglycol-2000)-1,2-dimyristoyl-sn-glycero-3-phosphoethanolamine (sodium salt; DMPE-MPEG-2000); N-(Carbonyl-methoxypolyethyleneglycol-5000)-1,2-dimyristoyl-sn-glycero-3-phosphoethanolamine (sodium salt; DMPE-MPEG-5000); N-(Carbonyl-methoxypolyethyleneglycol 2000)-1,2-dipalmitoyl-sn-glycero-3-phosphoethanolamine (sodium salt; DPPE-MPEG-2000); N-(Carbonyl-methoxypolyethyleneglycol 5000)-1,2-dipalmitoyl-sn-glycero-3-phosphoethanolamine (sodium salt; DPPE-MPEG-5000); N-(Carbonyl-methoxypolyethyleneglycol 750)-1,2-distearoyl-sn-glycero-3-phosphoethanolamine (sodium salt; DSPE-MPEG-750); N-(Carbonyl-methoxypolyethyleneglycol 2000)-1,2-distearoyl-sn-glycero-3-phosphoethanolamine (sodium salt; DSPE-MPEG-2000); N-(Carbonyl-methoxypolyethyleneglycol 5000)-1,2-distearoyl-sn-glycero-3-phosphoethanolamine (sodium salt; DSPE-MPEG-5000); sodium cholesteryl sulfate (SCS); pharmaceutically acceptable salts thereof, and mixtures thereof.

Examples of non-cationic lipids include diolcoylphosphatidylethanolamine (DOPE), diphytanoylphosphatidylethanolamine (DPhPE), 1,2-Dioleoyl-sn-Glycero-3-Phosphocholine (DOPC), 1,2-Diphytanoyl-sn-Glycero-3-Phosphocholine (DPhPC), cholesterol, and mixtures thereof.

The chemical structure of 1,2-Dipalmitoyl-sn-Glycero-3-Phosphocholine (DPPC) is as follows:

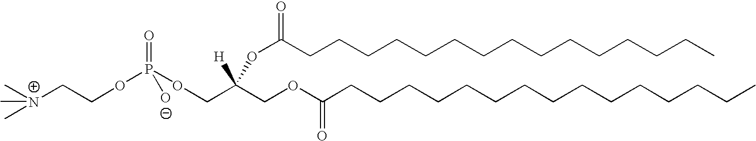

The chemical structure of 1,2-Distearoyl-sn-Glycero-3-Phosphocholine (DSPC) is as follows:

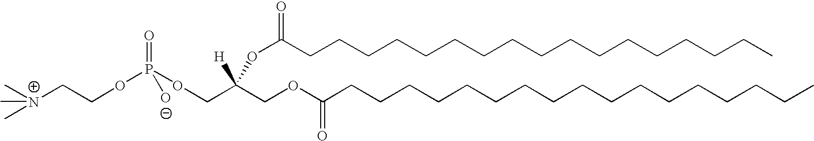

The chemical structure of 1,2-Distearoyl-sn-Glycero-3-Phosphoethanolamine (DSPE) is as follows:

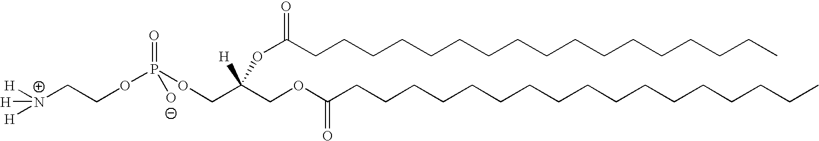

The chemical structure of 1,2-Dimyristoylamido-1,2-Deoxyphosphatidyl Choline (DDPC) is as follows:

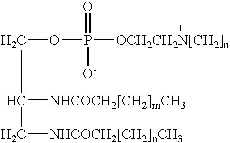

The chemical structure of 1,2-Dioleoyl-sn-Glycero-3-Phosphoethanolamine (DOPE) is as follows:

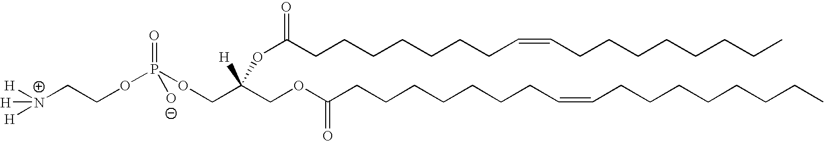

The chemical structure of 1,2-Dioleoyl-sn-Glycero-3-Phosphocholine (DOPC) is as follows:

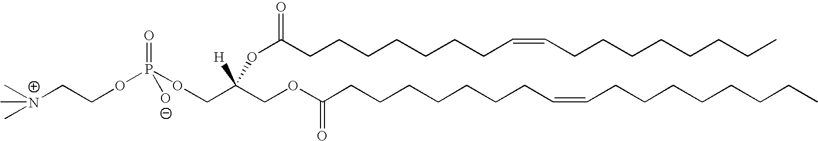

The chemical structure of 1,2-Diphytanoyl-sn-Glycero-3-Phosphocholine (DPhPC) is as follows:

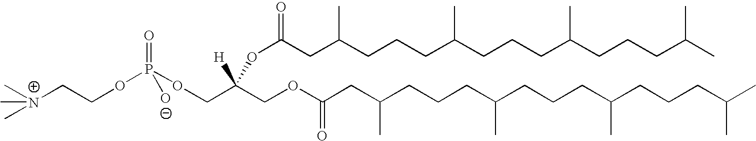

Examples of non-cationic lipids include lipids ranging from C10:0 to C22:6 Phosphoethanolamine as shown in Table 2.

TABLE 2
Examples of Non-cationic Lipids

| Name | FA chains | M.W. | CAS Registry # |
|---|---|---|---|
| DDPE | C10:0 | 523.64 | 253685-27-7 |
| DLPE | C12:0 | 579.76 | 59752-57-7 |
| DSPE | C18:0 | 748.08 | 1069-79-0 |
| DOPE | C18:1 | 744.05 | 4004-05-1 |
| DLinPE | C18:2 | 740.01 | 20707-71-5 |
| DLenPE | C18:3 | 735.98 | 34813-40-6 |
| DARAPE | C20:4 | 788.06 | 5634-86-6 |
| DDHAPE | C22:6 | 836.10 | 123284-81-1 |
| DPhPE | 16:0[(CH3)4] | 804.19 | 201036-16-0 |

Examples of anionic lipids include phosphatidylserine, phosphatidic acid, phosphatidylcholine, platelet-activation factor (PAF), phosphatidylethanolamine, phosphatidyl-DL-glycerol, phosphatidylinositol, phosphatidylinositol (pi(4)p, pi(4,5)p2), cardiolipin (sodium salt), lysophosphatides, hydrogenated phospholipids, sphingoplipids, gangliosides, phytosphingosine, sphinganines, pharmaceutically acceptable salts thereof, and mixtures thereof.

The chemical structure of 1,2-Dipalmitoyl-sn-Glycero-3-[Phospho-rac-(1-glycerol)] (sodium salt; DPPG) is as follows:

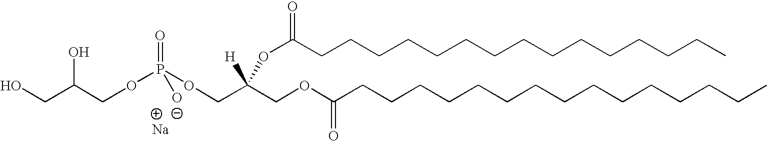

The chemical structure of 1,2-Dimyristoyl-sn-Glycero-3-[Phospho-rac-(1-glycerol)] (sodium salt; DMPG) is as follows: 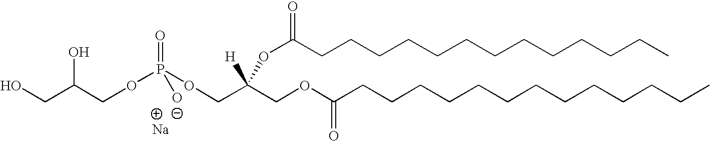
Delivery-Enhancing Lipophilic Compounds

Delivery-enhancing lipophilic components of compositions of this invention include omega-3 oils and lipid-soluble compounds such as tocopherols and Vitamin E.

Omega-3 fatty acids are a class of polyunsaturated fatty acids having an unsaturated carbon in the ω-3 position. Omega-3 fatty acids include α-linolenic acid (ALA), eicosapentaenoic acid (EPA), and docosahexaenoic acid (DHA).

Examples of omega-3 fatty acids are shown in Table 3.

TABLE 3
Example Omega-3 Fatty Acids

| | | |
|---|---|---|
| α-Linolenic acid | 18:3 (n-3) | octadeca-9,12,15-trienoic acid |
| (ALA) | | |
| Stearidonic acid | 18:4 (n-3) | octadeca-6,9,12,15-tetraenoic acid |
| Eicosatetraenoic acid | 20:4 (n-3) | eicosa-8,11,14,17-tetraenoic acid |
| Eicosapentaenoic acid | 20:5 (n-3) | eicosa-5,8,11,14,17-pentaenoic acid |
| (EPA) | | |
| Docosapentaenoic acid | 22:5 (n-3) | docosa-7,10,13,16,19-pentaenoic acid |
| Docosahexaenoic acid | 22:6 (n-3) | docosa-4,7,10,13,16,19-hexaenoic |
| (DHA) | | acid |

Delivery-enhancing lipophilic components of compositions of this invention include Vitamin E and related tocol (2-methyl-2-(4,8,12-trimethyltridecyl)chroman-6-ol) and tocotrienol derivatives.

Delivery-enhancing lipophilic components of compositions of this invention include tocopherols which are mono, di, and trimethyltocols, such as α-tocopherol and 2-methyl-2-(4,8,12-trimethyltrideca-3,7,11-trienyl)chroman-6-ol (E,E)-tocotrienol), among other steroisomers.

Peptides

In some embodiments, the polypeptide (peptide) may contain a hydrophobic region and a positively charged region. The hydrophobic region may consist of a cluster of at least two hydrophobic residues, preferably two or more, more preferably three or more and most preferably four or more, that lie within a stretch of nine residues that excludes a positively charged residue. The positively charged region consists of a cluster of at least three positively charged residues, preferably three or more, more preferably four or more and most preferably five or more that lie within a stretch of ten residues or less.

In some embodiments of this disclosure, the peptide is selected or designed to include an amphipathic amino acid sequence. For example, useful polypeptides may be selected which comprise a plurality of non-polar or hydrophobic amino acid residues that form a hydrophobic sequence domain or motif, linked to a plurality of charged amino acid residues that form a charged sequence domain or motif, yielding an amphipathic peptide.

In some embodiments, the polypeptide may consist of a positively charged domain and have a hydrophobic region resulting from the presence of a covalently linked lipid moiety, for example a fatty acid.

In some embodiments, the polypeptide has the following amino acid sequence:

(SEQ ID NO: 54)

NH₂-KETWWETWWTEWSQPGRKKRRQRRRPPQ

A polypeptide of this disclosure may be pegylated to improve stability and/or efficacy, particularly in the context of in vivo administration, or for use in a medicament.

A polypeptide of this disclosure may be selected to include a protein transduction domain (PTD) or motif, and/or a fusogenic peptide domain or motif. A protein transduction domain is a peptide sequence that is able to insert into and preferably transit through the membrane of cells. A fusogenic peptide is a peptide that destabilizes a lipid membrane, for example a plasma membrane or membrane surrounding an endosome, which may be enhanced at low pH. Exemplary fusogenic domains or motifs are found in a broad diversity of viral fusion proteins and in other proteins, for example fibroblast growth factor 4 (FGF-4).

To design polypeptides of this disclosure, a protein transduction domain may be employed as a motif that will facilitate entry of the nucleic acid into a cell through the plasma membrane. In some embodiments, the transported nucleic acid will be encapsulated in an endosome. The interior of endosomes in general has a low pH resulting in the fusogenic peptide motif destabilizing the membrane of the endosome. The destabilization and breakdown of the endosome membrane allows for the release of the interfering RNA into the cytoplasm where it can associate with a RISC complex and be directed to its target mRNA.

Examples of protein transduction domains include:

(SEQ ID NO: 1) (TAT)
KRRQRRR;
(SEQ ID NO: 2) (Penetratin)
RQIKIWFQNRRMKWKK;
(SEQ ID NO: 3) (VP22)
DAATATRGRSAASRPTERPRAPARSASRPRRPVD;
(SEQ ID NO: 4) (Kaposi FGF signal sequences)
AAVALLPAVLLALLAP,
(SEQ ID NO: 5)
AAVLLPVLLPVLLAAP;
(SEQ ID NO: 6) (Human β3 integrin signal sequence)
VTVLALGALAGVGVG;
(SEQ ID NO: 7) (gp41 fusion sequence)
GALFLGWLGAAGSTMGA;
(SEQ ID NO: 8) (Caiman crocodylus Ig(v) light
chain)
MGLGLHLLVLAAALQGA;
(SEQ ID NO: 9) (hCT-derived peptide)
LGTYTQDFNKFHTFPQTAIGVGAP;
(SEQ ID NO: 10) (Transportan)
GWTLNSAGYLLKINLKALAALAKKIL;
(SEQ ID NO: 11) (Loligomer)
TPPKKKRKVEDPKKKK;
(SEQ ID NO: 12)
RRRRRRR;
(SEQ ID NO: 13) (amphiphilic peptide)
KLALKLALKALKAALKLA.

In some embodiments, the delivery peptide may be a fusogenic peptide domain or motif In general, a fusogenic peptide is a peptide that can impart fusion activity to a biological material towards a membrane or cell. A fusogenic peptide may destabilize a lipid membrane, for example a plasma membrane or membrane surrounding an endosome, thereby inducing fusion. Fusogenic activity may be greater at lower pH. Exemplary fusogenic domains or motifs are found in a broad diversity of viral fusion proteins and in other proteins, for example, fibroblast growth factor 4 (FGF-4).

Examples of fusogenic domains include:

(SEQ ID NO: 14) (Influenza HA2)
GLFGAIAGFIENGWEG;
(SEQ ID NO: 15) (Sendai F1)
FFGAVIGTIALGVATA;
(SEQ ID NO: 16) (Respiratory Syncytial virus F1)
FLGFLLGVGSAIASGV;
(SEQ ID NO: 17) (HIV gp41)
GVFVLGFLGFLATAGS;
(SEQ ID NO: 18) (Ebola GP2)
GAAIGLAWIPYFGPAA.

A protein transduction domain may be employed as a motif to facilitate entry of a nucleic acid agent into a cell through a plasma membrane. In some embodiments, a nucleic acid agent may be encapsulated in an endosome. The interior of an endosome may have a low pH so that a fusogenic peptide motif may destabilize the membrane of the endosome to allow release of the nucleic acid agent.

In some embodiments, the delivery peptide may include a nucleic acid binding domain or motif. Exemplary DNA binding domains include various “zinc finger” domains as described for DNA-binding regulatory proteins and other proteins identified in Table 4, below (see, e.g., Simpson, et al., J. Biol. Chem. 278:28011-28018, 2003).

TABLE 4

Exemplary Zinc Finger Motifs of DNA-binding Proteins
C₂H₂ Zinc finger motif

| | | | | | | |
|---|---|---|---|---|---|---|
| | ....\|....\| | ....\|....\| | ....\|....\| | ....\|....\| | ....\|....\| | ....\|....\| |
| | 665 | 675 | 685 | 695 | 705 | 715 |
| Sp1 | ACTCPYCKDS | EGRGSG---- | DPGKKKQHIC | HIQGCGKVYG | KTSHLRAHLR | WHTGERPFMC |
| Sp2 | ACTCPNCKDG | EKRS------ | GEQGKKKHVC | HIPDCGKTFR | KTSLLRAHVR | LHTGERPFVC |
| Sp3 | ACTCPNCKEG | GGRGTN---- | -LGKKKQHIC | HIPGCGKVYG | KTSHLRAHLR | WHSGERPFVC |
| Sp4 | ACSCPNCREG | EGRGSN---- | EPGKKKQHIC | HIEGCGKVYG | KTSHLRAHLR | WHTGERPFIC |
| DrosBtd | RCTCPNCTNE | MSGLPPIVGP | DERGRKQHIC | HIPGCERLYG | KASHLKTHLR | WHTGERPFLC |
| DrosSp | TCDCPNCQEA | ERLGPAGV-- | HLRKKNIHSC | HIPGCGKVYG | KTSHLKAHLR | WHTGERPFVC |
| CeT22C8.5 | RCTCPNCKAI | KHG------- | DRGSQHTHLC | SVPGCGKTYK | KTSHLRAHLR | KHTGDRPFVC |
| Y40B1A.4 | PQISLKKKIF | FFIFSNFR-- | GDGKSRIHIC | HL--CNKTYG | KTSHLRAHLR | GHAGNKPFAC |

The sequences shown in Table 4 for Sp1, Sp2, Sp3, Sp4, DrosBtd, DrosSp, CeT22C8.5, and Y40B1A.4, are herein assigned SEQ ID NOs: 19, 20, 21, 22, 23, 24, 25, and 26, respectively.

Table 4 shows a conservative zinc finger motif for double strand DNA binding which is characterized by the PROSITE pattern C-x(2,4)-C-x(12)-H-x(3)-H (SEQ ID NO: 55) motif. This motif can also be used to select and design additional polynucleotide delivery-enhancing polypeptides.

Alternative DNA binding domains useful for constructing polynucleotide delivery-enhancing polypeptides of the invention include, for example, portions of the HIV TAT protein sequence.

Within exemplary embodiments of the invention described herein below, polypeptides may be rationally designed and constructed by combining any of the foregoing structural elements, domains or motifs into a single polypeptide effective to mediate enhanced delivery of interfering RNA into target cells. For example, a protein transduction domain of the TAT polypeptide was fused to the N-terminal 20 amino acids of the influenza virus hemagglutinin protein, termed HA2, to yield one exemplary polypeptide herein. Various other polypeptide constructs are provided in the instant disclosure, evincing that the concepts of the invention are broadly applicable to create and use a diverse assemblage of effective polypeptides for enhancing RNA delivery.

Yet additional exemplary polypeptides within the invention may be selected from the following peptides:

(SEQ ID NO: 27)

WWETWKPFQCRICMRNFSTRQARRNHRRRHR

(SEQ ID NO: 28)

GKINLKALAALAKKIL,

(SEQ ID NO: 29)

RVIRVWFQNKRCKDKK,

(SEQ ID NO: 30)

GRKKRRQRRRPPQGRKKRRQRRRPPQGRKKRRQRRRPPQ

(SEQ ID NO: 31)

GEQIAQLIAGYIDIILKKKKSK

Additional polypeptides that are useful within the compositions and methods disclosed herein include Poly Lys-Trp, 4:1, MW 20,000-50,000 and Poly Orn-Trp, 4:1, MW 20,000-50,000.

Additional polypeptides that are useful within the compositions and methods herein comprise all or part of the mellitin protein sequence.

Still other exemplary polypeptides are identified in the examples below. Any one or combination of these peptides may be selected or combined to yield effective polypeptide reagents to induce or facilitate intracellular delivery of siNAs within the methods and compositions of the invention.

Compositions and Formulations for Administration

The nucleic acid-lipid compositions of this invention may be administered by various routes, for example, to effect systemic delivery via intravenous, parenteral, or intraperitoneal routes. In some embodiments, an siRNA may be delivered intracellularly, for example, in cells of a target tissue such as lung or liver, or in inflamed tissues. In some embodiments, this invention provides a method for delivery of siRNA in vivo. A nucleic acid-lipid composition may be administered intravenously, subcutaneously, or intraperitoneally to a subject. In some embodiments, the invention provides methods for in vivo delivery of interfering RNA to the lung of a mammalian subject.

In some embodiments, this invention provides a method of treating a disease or disorder in a mammalian subject. A therapeutically effective amount of a composition of this invention containing an RNA, a cationic lipid, a non-cationic lipid, and one or more delivery-enhancing lipophilic components may be administered to a subject having a disease or disorder associated with expression or overexpression of a gene that can be reduced, decreased, downregulated, or silenced by the composition.

This invention encompasses a method for treating a disease of the lung such as respiratory distress, asthma, cystic fibrosis, pulmonary fibrosis, chronic obstructive pulmonary disease, bronchitis, or emphysema, by administering to the subject a therapeutically effective amount of a composition.

This invention encompasses methods for treating rheumatoid arthritis, liver disease, encephalitis, bone fracture, heart disease, viral disease including hepatitis and influenza, or cancer.

The compositions and methods of the invention may be administered to subjects by a variety of mucosal administration modes, including by oral, rectal, vaginal, intranasal, intrapulmonary, or transdermal delivery, or by topical delivery to the eyes, ears, skin or other mucosal surfaces. In some aspects of this invention, the mucosal tissue layer includes an epithelial cell layer. The epithelial cell can be pulmonary, tracheal, bronchial, alveolar, nasal, buccal, epidermal, or gastrointestinal. Compositions of this invention can be administered using conventional actuators such as mechanical spray devices, as well as pressurized, electrically activated, or other types of actuators.

Compositions of this invention may be administered in an aqueous solution as a nasal or pulmonary spray and may be dispensed in spray form by a variety of methods known to those skilled in the art. Pulmonary delivery of a composition of this invention is achieved by administering the composition in the form of drops, particles, or spray, which can be, for example, aerosolized, atomized, or nebulized. Particles of the composition, spray, or aerosol can be in a either liquid or solid form. Preferred systems for dispensing liquids as a nasal spray are disclosed in U.S. Pat. No. 4,511,069. Such formulations may be conveniently prepared by dissolving compositions according to the present invention in water to produce an aqueous solution, and rendering said solution sterile. The formulations may be presented in multi-dose containers, for example in the sealed dispensing system disclosed in U.S. Pat. No. 4,511,069. Other suitable nasal spray delivery systems have been described in Transdermal Systemic Medication, Y. W. Chien ed., Elsevier Publishers, New York, 1985; and in U.S. Pat. No. 4,778,810. Additional aerosol delivery forms may include, e.g., compressed air-, jet-, ultrasonic-, and piezoelectric nebulizers, which deliver the biologically active agent dissolved or suspended in a pharmaceutical solvent, e.g., water, ethanol, or mixtures thereof.

Nasal and pulmonary spray solutions of the present invention typically comprise the drug or drug to be delivered, optionally formulated with a surface active agent, such as a nonionic surfactant (e.g., polysorbate-80), and one or more buffers. In some embodiments of the present invention, the nasal spray solution further comprises a propellant. The pH of the nasal spray solution may be from about pH 6.8 to 7.2. The pharmaceutical solvents employed can also be a slightly acidic aqueous buffer of pH 4-6. Other components may be added to enhance or maintain chemical stability, including preservatives, surfactants, dispersants, or gases.

In some embodiments, this invention is a pharmaceutical product which includes a solution containing a composition of this invention and an actuator for a pulmonary, mucosal, or intranasal spray or aerosol.

A dosage form of the composition of this invention can be liquid, in the form of droplets or an emulsion, or in the form of an aerosol.

A dosage form of the composition of this invention can be solid, which can be reconstituted in a liquid prior to administration. The solid can be administered as a powder. The solid can be in the form of a capsule, tablet or gel.

To formulate compositions for pulmonary delivery within the present invention, the biologically active agent can be combined with various pharmaceutically acceptable additives, as well as a base or carrier for dispersion of the active agent(s). Examples of additives include pH control agents such as arginine, sodium hydroxide, glycine, hydrochloric acid, citric acid, and mixtures thereof. Other additives include local anesthetics (e.g., benzyl alcohol), isotonizing agents (e.g., sodium chloride, mannitol, sorbitol), adsorption inhibitors (e.g., Tween 80), solubility enhancing agents (e.g., cyclodextrins and derivatives thereof), stabilizers (e.g., serum albumin), and reducing agents (e.g., glutathione). When the composition for mucosal delivery is a liquid, the tonicity of the formulation, as measured with reference to the tonicity of 0.9% (w/v) physiological saline solution taken as unity, is typically adjusted to a value at which no substantial, irreversible tissue damage will be induced in the mucosa at the site of administration. Generally, the tonicity of the solution is adjusted to a value of about ⅓ to 3, more typically ½ to 2, and most often ¾ to 1.7.

The biologically active agent may be dispersed in a base or vehicle, which may comprise a hydrophilic compound having a capacity to disperse the active agent and any desired additives. The base may be selected from a wide range of suitable carriers, including but not limited to, copolymers of polycarboxylic acids or salts thereof, carboxylic anhydrides (e.g. maleic anhydride) with other monomers (e.g., methyl(meth)acrylate, acrylic acid, etc.), hydrophilic vinyl polymers such as polyvinyl acetate, polyvinyl alcohol, polyvinylpyrrolidone, cellulose derivatives such as hydroxymethylcellulose, hydroxypropylcellulose, etc., and natural polymers such as chitosan, collagen, sodium alginate, gelatin, hyaluronic acid, and nontoxic metal salts thereof. Often, a biodegradable polymer is selected as a base or carrier, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters 4such as polyglycerin fatty acid esters, sucrose fatty acid esters, etc., can be employed as carriers. Hydrophilic polymers and other carriers can be used alone or in combination, and enhanced structural integrity can be imparted to the carrier by partial crystallization, ionic bonding, crosslinking and the like. The carrier can be provided in a variety of forms, including, fluid or viscous solutions, gels, pastes, powders, microspheres and films for direct application to the nasal mucosa. The use of a selected carrier in this context may result in promotion of absorption of the biologically active agent.

The biologically active agent can be combined with the base or carrier according to a variety of methods, and release of the active agent may be by diffusion, disintegration of the carrier, or associated formulation of water channels. In some circumstances, the active agent is dispersed in microcapsules (microspheres) or nanocapsules (nanospheres) prepared from a suitable polymer, e.g., isobutyl 2-cyanoacrylate (see, e.g., Michael, et al., J. Pharmacy Pharmacol. 43:1-5, 199 1), and dispersed in a biocompatible dispersing medium applied to the nasal mucosa, which yields sustained delivery and biological activity over a protracted time.

Formulations for mucosal, nasal, or pulmonary delivery may contain a hydrophilic low molecular weight compound as a base or excipient. Such hydrophilic low molecular weight compounds provide a passage medium through which a water-soluble active agent, such as a physiologically active peptide or protein, may diffuse through the base to the body surface where the active agent is absorbed. The hydrophilic low molecular weight compound optionally absorbs moisture from the mucosa or the administration atmosphere and dissolves the water-soluble active peptide. The molecular weight of the hydrophilic low molecular weight compound is generally not more than 10,000 and preferably not more than 3000. Examples of hydrophilic low molecular weight compounds include polyol compounds, such as oligo-, di- and monosaccarides including sucrose, mannitol, lactose, L-arabinose, D-erythrose, D-ribose, D-xylose, D-mannose, D-galactose, lactulose, cellobiose, gentibiose, glycerin, polyethylene glycol, and mixtures thereof. Further examples of hydrophilic low molecular weight compounds include N-methylpyrrolidone, alcohols (e.g., oligovinyl alcohol, ethanol, ethylene glycol, propylene glycol, etc.), and mixtures thereof.

The compositions of this invention may alternatively contain as pharmaceutically acceptable carriers substances as required to approximate physiological conditions, such as pH adjusting and buffering agents, tonicity adjusting agents, and wetting agents, for example, sodium acetate, sodium lactate, sodium chloride, potassium chloride, calcium chloride, sorbitan monolaurate, triethanolamine oleate, and mixtures thereof. For solid compositions, conventional nontoxic pharmaceutically acceptable carriers can be used which include, for example, pharmaceutical grades of mannitol, lactose, starch, magnesium stearate, sodium saccharin, talcum, cellulose, glucose, sucrose, magnesium carbonate, and the like.

In certain embodiments of the invention, the biologically active agent may be administered in a time release formulation, for example in a composition which includes a slow release polymer. The active agent can be prepared with carriers that will protect against rapid release, for example a controlled release vehicle such as a polymer, microencapsulated delivery system or bioadhesive gel. Prolonged delivery of the active agent, in various compositions of the invention can be brought about by including in the composition agents that delay absorption, for example, aluminum monosterate hydrogels and gelatin.

Within certain embodiments of this invention, the siNA composition may contain one or more natural or synthetic surfactants. Certain natural surfactants are found in human lung (pulmonary surfactant), and are a complex mixture of phospholipids and proteins that form a monolayer at the alveolar air-liquid interface and reduces surface tension to near zero at expiration and prevents alveolar collapse. Over 90% (by weight) of pulmonary surfactant is composed of phospholipids with approximately 40-80% being DPPC and the remainder being unsaturated phosphatidylcholines POPG, POPC and phosphatidylglycerols. The remaining 10% (by weight) of surfactant is composed of plasma proteins and apoproteins, such as surface proteins (SP)-A, SP-B, SP-C and SP-D.

Examples of natural surfactants that may be used in this invention include SURVANTA™ (beractant), CUROSURF™ (poractant alfa) and INFASURF™ (calfactant), and mixtures thereof.

Examples of synthetic surfactants include sinapultide; a combination of dipalmitoylphosphatidylcholine, palmitoyloleoyl phosphatidylglycerol and palmitic acid; SURFAXIN™ (lucinactant); and EXOSURF™ (colfosceril); components which may contain tyloxapol, DPPC, and hexadecanol; and mixtures thereof.

Compositions of this invention can be prepared by methods known in the art. Methods of making the lipid compositions include ethanol injection methods and extrusion methods using a Northern Lipids Lipex Extruder system with stacked polycarbonate membrane filters of defined pore size. Sonication using probe tip and bath sonicators can be employed to produce lipid particles of uniform size. Homogenous and monodisperse particle sizes can be obtained without the addition of the nucleic acid component. For in vitro transfection compositions, the nucleic acid component can be added after the transfection agent is made and stabilized by additional buffer components. For in vivo delivery compositions, the nucleic acid component is part of the formulation.

A mixing procedure involving the graded substitution of ethanol for buffer can be used to create a narrow homodisperse particle size distribution. The lipid components can be dissolved in USP absolute ethanol and the RNA component can be dissolved in an aqueous buffer, for example, at 0.9 mg/mL. Both mixtures can be injected through an HPLC mixing tee into a 20 mM citrate buffer at pH 7.2. The initial concentration of ethanol may be 90% for the lipids and 0% for the RNA component. After mixing into a 45% ethanol mixture, the RNA-lipid particles can be immediately diluted into citrate buffer with a final concentration of ethanol at 30%. The ethanol and citrate buffer can be exchanged for PBS, pH 7.2 by overnight dialysis in a Pierce dialysis cassette with a 2K MWCO membrane. Particle sizing and PAGE gel analysis of each siRNA lipid particle can be performed to confirm successful entrapment. PAGE gel results can confirm that an siRNA is intact after exposure to multiple processing steps.

The nucleic acid component and cationic lipid may be mixed together first in a suitable medium such as a cell culture medium, after which one or more additional lipids may be added to the mixture. Alternatively, the lipids can be mixed together first in a suitable medium such as a cell culture medium, after which the nucleic acid component can be added.

Within certain embodiments of the invention, an interfering RNA is admixed with one or more non-cationic lipids, or a combination of one or more non-cationic and cationic lipids.

The interfering RNA may also be complexed with a lipid, peptide, or polymer, and admixed with one or more non-cationic lipids, or peptides, or a combination of one or more non-cationic and cationic lipids.

An interfering RNA and a lipid component may be mixed together first, followed by the addition of one or more non-cationic lipids, or a combination of non-cationic and cationic lipids added in a suitable medium such as a cell culture medium. Alternatively, the lipid components may be mixed first, followed by the addition of the siNA in a suitable medium.

RNA Therapeutics and RNA Interference

This invention provides compositions and methods for modulating gene expression by RNA interference. A composition of this invention can deliver a ribonucleic acid agent to a cell which can produce the response of RNAi. Examples of nucleic acid agents useful for this invention include double-stranded nucleic acids, modified or degradation-resistant nucleic acids, RNA, siRNA, siNA, shRNA, single-stranded nucleic acids, DNA-RNA chimeras, antisense nucleic acids, and ribozymes.

Ribonucleic acid agents useful for this invention may be targeted to various genes. For example, a siRNA agent of this invention may have a sequence that is complementary to a region of a TNF-alpha gene. In some embodiments of this invention, compounds and compositions are useful to regulate expression of tumor necrosis factor-α (TNF-α). TNF-α can be linked, for example, to inflammatory processes which occur in pulmonary diseases, and can have anti-inflammatory effects. Blocking TNF-α by delivery of a composition of this invention can be useful to treat or prevent the signs and/or symptoms of rheumatoid arthritis. This invention provides compositions and methods for modulating expression and activity of TNF-α by RNA interference.

Expression and/or activity of TNF-α can be modulated by delivering to a cell, for example, the siRNA molecule Inm-4. Inm-4 is a double stranded 21-nt siRNA molecule with sequence homology to the mouse TNF-α gene. Inm-4 has a 3′ dTdT overhang on the sense strand and a 3′ dAdT overhang on the antisense strand. The primary structure of Inm-4 is:

(SEQ ID NO: 32) sense
5′-CCGUCAGCCGAUUUGCUAUdTdT
(SEQ ID NO: 33) antisense
5′-AUAGCAAAUCGGCUGACGGdTdT

Expression and/or activity of TNF-α can be modulated by delivering to a cell, for example, the siRNA molecule LC20. LC20 is a double stranded 21-nt siRNA molecule with sequence homology to the human TNF-α gene. LC20 is directed against the 3′-UTR region of human TNF-α. LC 20 has 19 base pairs with a 3′ dTdT overhang on the sense strand and a 3′ dAdT overhang on the antisense strand. The molecular weight of the sodium salt form is 14,298. The primary structure of LC20 is:

(SEQ ID NO: 34) sense
(5′) GGGUCGGAACCCAAGCUUAdTdT
(SEQ ID NO: 35) antisense
(5′) UAAGCUUGGGUUCCGACCCdTdA

A beta-galactoside reporter cell line was used to assay the RNAi activity of various formulations. The structure of Lac-Z is:

Sense: CN2938. (SEQ ID NO: 36)
5′-CUACACAAAUCAGCGAUUUdTdT-3′
Antisense: CN2939. (SEQ ID NO: 37)
5′-AAAUCGCUGAUUUGUGUAGdTdC-3′

A siRNA of this invention may have a sequence that is complementary to a region of a viral gene. For example, some compositions and methods of this invention are useful to regulate expression of the viral genome of an influenza.

In this context, this invention provides compositions and methods for modulating expression and infectious activity of an influenza by RNA interference. Expression and/or activity of an influenza can be modulated by delivering to a cell, for example, a short interfering RNA molecule having a sequence that is complementary to a region of a RNA polymerase subunit of an influenza. For example, in Table 5 are shown double-stranded siRNA molecules with sequence homology to an RNA polymerase subunit of an influenza.

TABLE 5

Double-Stranded siRNA Molecules Targeted to
Influenza

| | | |
|---|---|---|
| | Sub- | |
| siRNA | unit | SEQUENCE |

| | | | |
|---|---|---|---|
| G3789 | PB2 | (SEQ ID NO 38) | CGGGACUCUAGCAUACUUAdTdT |
| | | (SEQ ID NO 39) | UAAGUAUGCUAGAGUCCCGdTdT |
| G3807 | PB2 | (SEQ ID NO 40) | ACUGACAGCCAGACAGCGAdTdT |
| | | (SEQ ID NO 41) | UCGCUGUCUGGCUGUCAGUdTdT |
| G3817 | PB2 | (SEQ ID NO 42) | AGACAGCGACCAAAAGAAUdTdT |
| | | (SEQ ID NO 43) | AUUCUUUUGGUCGCUGUCUdTdT |
| G6124 | PB1 | (SEQ ID NO 44) | AUGAAGAUCUGUUCCACCAdTdT |
| | | (SEQ ID NO 45) | UGGUGGAACAGAUCUUCAUdTdT |
| G6129 | PB1 | (SEQ ID NO 46) | GAUCUGUUCCACCAUUGAAdTdT |
| | | (SEQ ID NO 47) | UUCAAUGGUGGAACAGAUCdTdT |
| G8282 | PA | (SEQ ID NO 48) | GCAAUUGAGGAGUGCCUGAdTdT |
| | | (SEQ ID NO 49) | UCAGGCACUCCUCAAUUGCdTdT |
| G8286 | PA | (SEQ ID NO 50) | UUGAGGAGUGCCUGAUUAAdTdT |
| | | (SEQ ID NO 51) | UUAAUCAGGCACUCCUCAAdTdT |
| G1498 | NP | (SEQ ID NO 52) | GGAUCUUAUUUCUUCGGAGdTdT |
| | | (SEQ ID NO 53) | CUCCGAAGAAAUAAGAUCCdTdT |

A siRNA of this invention may have a sequence that is complementary to a region of a RNA polymerase subunit of an influenza.

This invention provides compositions and methods to administer siNAs directed against a mRNA of an influenza, which effectively down-regulates an influenza RNA and thereby reduces, prevents, or ameliorates an influenza infection.

In some embodiments, this invention provides compositions and methods for inhibiting expression of a target transcript in a subject by administering to the subject a composition containing an effective amount of an RNAi-inducing compound such as a short interfering oligonucleotide molecule, or a precursor thereof. RNAi uses small interfering RNAs (siRNAs) to target messenger RNA (mRNAs) and attenuate translation. A siRNA as used in this invention may be a precursor for dicer processing such as, for example, a long dsRNA processed into a siRNA. This invention provides methods of treating or preventing diseases or conditions associated with expression of a target transcript or activity of a peptide or protein encoded by the target transcript.

A therapeutic strategy based on RNAi can be used to treat a wide range of diseases by shutting down the growth or function of a virus or microorganism, as well as by shutting down the function of an endogenous gene product in the pathway of the disease.

In some embodiments, this invention provides novel compositions and methods for delivery of RNAi-inducing entities such as short interfering oligonucleotide molecules, and precursors thereof. In particular, this invention provides compositions containing an RNAi-inducing entity which is targeted to one or more transcripts of a cell, tissue, and/or organ of a subject.

A siRNA can be two RNA strands having a region of complementarity about 19 nucleotides in length. A siRNA optionally includes one or two single-stranded overhangs or loops.

A shRNA can be a single RNA strand having a region of self-complementarity. The single RNA strand may form a hairpin structure with a stem and loop and, optionally, one or more unpaired portions at the 5′ and/or 3′ portion of the RNA.

The active therapeutic agent can be a chemically-modified siNA with improved resistance to nuclease degradation in vivo, and/or improved cellular uptake, which retains RNAi activity.

A siRNA agent of this invention may have a sequence that is complementary to a region of a target gene. A siRNA of this invention may have 29-50 base pairs, for example, a dsRNA having a sequence that is complementary to a region of a target gene. Alternately, the double-stranded nucleic acid can be a dsDNA.

In some embodiments, the active agent can be a short interfering nucleic acid (siNA), short interfering RNA (siRNA), double-stranded RNA (dsRNA), micro-RNA, or short hairpin RNA (shRNA) that can modulate expression of a gene product.

Comparable methods and compositions are provided that target expression of one or more different genes associated with a particular disease condition in a subject, including any of a large number of genes whose expression is known to be aberrantly increased as a causal or contributing factor associated with the selected disease condition.

The RNAi-inducing compound of this invention can be administered in conjunction with other known treatments for a disease condition.

In some embodiments, this invention features compositions containing a small nucleic acid molecule, such as short interfering nucleic acid, a short interfering RNA, a double-stranded RNA, a micro-RNA, or a short hairpin RNA, admixed or complexed with, or conjugated to, a delivery-enhancing compound.

As used herein, the terms “short interfering nucleic acid,” “siNA,” “short interfering RNA,” “siRNA,” “short interfering nucleic acid molecule,” “short interfering oligonucleotide molecule,” and “chemically-modified short interfering nucleic acid molecule,” refer to any nucleic acid molecule capable of inhibiting or down regulating gene expression or viral replication, for example, by mediating RNA interference (RNAi) or gene silencing in a sequence-specific manner.

In some embodiments, the siNA is a double-stranded polynucleotide molecule comprising self-complementary sense and antisense regions, wherein the antisense region comprises a nucleotide sequence that is complementary to a nucleotide sequence in a target ribonucleic acid molecule for down regulating expression, or a portion thereof, and the sense region comprises a nucleotide sequence corresponding to (i.e., which is substantially identical in sequence to) the target ribonucleic acid sequence or portion thereof.

“siNA” means a small interfering nucleic acid, for example a siRNA, that is a short-length double-stranded nucleic acid, or optionally a longer precursor thereof. The length of useful siNAs within this invention will in some embodiments be optimized at a length of approximately 20 to 50 bp. However, there is no particular limitation to the length of useful siNAs, including siRNAs. For example, siNAs can initially be presented to cells in a precursor form that is substantially different than a final or processed form of the siNA that will exist and exert gene silencing activity upon delivery, or after delivery, to the target cell. Precursor forms of siNAs may, for example, include precursor sequence elements that are processed, degraded, altered, or cleaved at or after the time of delivery to yield a siNA that is active within the cell to mediate gene silencing. In some embodiments, useful siNAs will have a precursor length, for example, of approximately 100-200 base pairs, or 50-100 base pairs, or less than about 50 base pairs, which will yield an active, processed siNA within the target cell. In other embodiments, a useful siNA or siNA precursor will be approximately 10 to 49 bp, or 15 to 35 bp, or about 21 to 30 bp in length.

In some embodiments of this invention, polynucleotide delivery-enhancing polypeptides are used to facilitate delivery of larger nucleic acid molecules than conventional siNAs, including large nucleic acid precursors of siNAs. For example, the methods and compositions herein may be employed for enhancing delivery of larger nucleic acids that represent “precursors” to desired siNAs, wherein the precursor amino acids may be cleaved or otherwise processed before, during or after delivery to a target cell to form an active siNA for modulating gene expression within the target cell. 4

For example, a siNA precursor polynucleotide may be selected as a circular, single-stranded polynucleotide, having two or more loop structures and a stem comprising self-complementary sense and antisense regions, wherein the antisense region comprises a nucleotide sequence that is complementary to a nucleotide sequence in a target nucleic acid molecule or a portion thereof, and the sense region having nucleotide sequence corresponding to the target nucleic acid sequence or a portion thereof, and wherein the circular polynucleotide can be processed either in vivo or in vitro to generate an active siNA molecule capable of mediating RNAi.

siNA molecules of this invention, particularly non-precursor forms, can be less than 30 base pairs, or about 17-19 bp, or 19-21 bp, or 21-23 bp.

siRNAs can mediate selective gene silencing in the mammalian system. Hairpin RNAs, with a short loop and 19 to 27 base pairs in the stem, also selectively silence expression of genes that are homologous to the sequence in the double-stranded stem. Mammalian cells can convert short hairpin RNA into siRNA to mediate selective gene silencing.

RISC mediates cleavage of single stranded RNA having sequence complementary to the antisense strand of the siRNA duplex. Cleavage of the target RNA takes place within the region complementary to the antisense strand of the siRNA duplex. siRNA duplexes of 21 nucleotides are typically most active when containing two-nucleotide 3′-overhangs.

Replacing the 3′-overhanging segments of a 21-mer siRNA duplex having 2-nucleotide 3′ overhangs with deoxyribonucleotides may not have an adverse effect on RNAi activity. Replacing up to 4 nucleotides on each end of the siRNA with deoxyribonucleotides can be tolerated whereas complete substitution with deoxyribonucleotides may result in no RNAi activity.

Alternatively, the siNAs can be delivered as single or multiple transcription products expressed by a polynucleotide vector encoding the single or multiple siNAs and directing their expression within target cells. In these embodiments the double-stranded portion of a final transcription product of the siRNAs to be expressed within the target cell can be, for example, 15 to 49 bp, 15 to 35 bp, or about 21 to 30 bp long.

In some embodiments of this invention, the double-stranded region of siNAs in which two strands are paired may contain bulge or mismatched portions, or both. Double-stranded portions of siNAs in which two strands are paired are not limited to completely paired nucleotide segments, and may contain nonpairing portions due to, for example, mismatch (the corresponding nucleotides not being complementary), bulge (lacking in the corresponding complementary nucleotide on one strand), or overhang. Nonpairing portions can be contained to the extent that they do not interfere with siNA formation. In some embodiments, a “bulge” may comprise 1 to 2 nonpairing nucleotides, and the double-stranded region of siNAs in which two strands pair up may contain from about 1 to 7, or about 1 to 5 bulges. In addition, “mismatch” portions contained in the double-stranded region of siNAs may be present in numbers from about 1 to 7, or about 1 to 5. Most often in the case of mismatches, one of the nucleotides is guanine, and the other is uracil. Such mismatching may be attributable, for example, to a mutation from C to T, G to A, or mixtures thereof, in a corresponding DNA coding for sense RNA, but other causes are also contemplated.

The terminal structure of siNAs of this invention may be either blunt or cohesive (overhanging) as long as the siNA retains its activity to silence expression of target genes. The cohesive (overhanging) end structure is not limited to the 3′ overhang, but includes the 5′ overhanging structure as long as it retains activity for inducing gene silencing. In addition, the number of overhanging nucleotides is not limited to 2 or 3 nucleotides, but can be any number of nucleotides as long as it retains activity for inducing gene silencing. For example, overhangs may comprise from 1 to about 8 nucleotides, or from 2 to 4 nucleotides.

The length of siNAs having cohesive (overhanging) end structure may be expressed in terms of the paired duplex portion and any overhanging portion at each end. For example, a 25/27-mer siNA duplex with a 2-bp 3′ antisense overhang has a 25-mer sense strand and a 27-mer antisense strand, where the paired portion has a length of 25 bp.

Any overhang sequence may have low specificity to a target gene, and may not be complementary (antisense) or identical (sense) to the target gene sequence. As long as the siNA retains activity for gene silencing, it may contain in the overhang portion a low molecular weight structure, for example, a natural RNA molecule such as a tRNA, an rRNA, a viral RNA, or an artificial RNA molecule.

The terminal structure of the siNAs may have a stem-loop structure in which ends of one side of the double-stranded nucleic acid are connected by a linker nucleic acid, e.g., a linker RNA. The length of the double-stranded region (stem portion of stem-loop structure) can be, for example, 15 to 49 bp, or 15 to 35 bp, or about 21 to 30 bp long. Alternatively, the length of the double-stranded region that is a final transcription product of siNAs to be expressed in a target cell may be, for example, approximately 15 to 49 bp, or 15 to 35 bp, or about 21 to 30 bp long.

The siNA can contain a single stranded polynucleotide having a nucleotide sequence complementary to a nucleotide sequence in a target nucleic acid molecule, or a portion thereof, wherein the single stranded polynucleotide can contain a terminal phosphate group, such as a 5′-phosphate (see, for example, Martinez, et al., Cell 110:563-574, 2002, and Schwarz, et al., Molecular Cell 10:537-568, 2002, or 5′,3′-diphosphate.

As used herein, the term siNA molecule is not limited to molecules containing only naturally-occurring RNA or DNA, but also encompasses chemically-modified nucleotides and non-nucleotides. In some embodiments, the short interfering nucleic acid molecules of the invention lack 2′-hydroxy (2′-OH) containing nucleotides. In some embodiments, short interfering nucleic acids do not require the presence of nucleotides having a 2′-hydroxy group for mediating RNAi and as such, short interfering nucleic acid molecules of this invention optionally do not include any ribonucleotides (e.g., nucleotides having a 2′-OH group). siNA molecules that do not require the presence of ribonucleotides within the siNA molecule to support RNAi can, however, have an attached linker or linkers or other attached or associated groups, moieties, or chains containing one or more nucleotides with 2′-OH groups. siNA molecules can comprise ribonucleotides in at least about 5, 10, 20, 30, 40, or 50% of the nucleotide positions.

As used herein, the term siNA encompasses nucleic acid molecules that are capable of mediating sequence specific RNAi such as, for example, short interfering RNA (siRNA) molecules, double-stranded RNA (dsRNA) molecules, micro-RNA molecules, short hairpin RNA (shRNA) molecules, short interfering oligonucleotide molecules, short interfering nucleic acid molecules, short interfering modified oligonucleotide molecules, chemically-modified siRNA molecules, and post-transcriptional gene silencing RNA (ptgsRNA) molecules, among others.

In some embodiments, siNA molecules comprise separate sense and antisense sequences or regions, wherein the sense and antisense regions are covalently linked by nucleotide or non-nucleotide linker molecules, or are non-covalently linked by ionic interactions, hydrogen bonding, van der waals interactions, hydrophobic interactions, and/or stacking interactions.

“Antisense RNA” is an RNA strand having a sequence complementary to a target gene mRNA, that can induce RNAi by binding to the target gene mRNA.

“Sense RNA” is an RNA strand having a sequence complementary to an antisense RNA, and anneals to its complementary antisense RNA to form a siRNA.

As used herein, the term “RNAi construct” or “RNAi precursor” refers to an RNAi-inducing compound such as small interfering RNAs (siRNAs), hairpin RNAs, and other RNA species which can be cleaved in vivo to form a siRNA. RNAi precursors herein also include expression vectors (also referred to as RNAi expression vectors) capable of giving rise to transcripts which form dsRNAs or hairpin RNAs in cells, and/or transcripts which can produce siRNAs in vivo.

A siHybrid molecule is a double-stranded nucleic acid that has a similar function to siRNA. Instead of a double-stranded RNA molecule, a siHybrid is comprised of an RNA strand and a DNA strand. Preferably, the RNA strand is the antisense strand which binds to a target mRNA. The siHybrid created by the hybridization of the DNA and RNA strands have a hybridized complementary portion and preferably at least one 3′overhanging end.

siNAs for use within the invention can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure, for example wherein the double stranded region is about 19 base pairs). The antisense strand may comprise a nucleotide sequence that is complementary to a nucleotide sequence in a target nucleic acid molecule or a portion thereof, and the sense strand may comprise a nucleotide sequence corresponding to the target nucleic acid sequence or a portion thereof. Alternatively, the siNA can be assembled from a single oligonucleotide, where the self-complementary sense and antisense regions of the siNA are linked by means of a nucleic acid-based or non-nucleic acid-based linker(s).

In some embodiments, siNAs for intracellular delivery can be a polynucleotide with a duplex, asymmetric duplex, hairpin or asymmetric hairpin secondary structure, having self-complementary sense and antisense regions, wherein the antisense region comprises a nucleotide sequence that is complementary to a nucleotide sequence in a separate target nucleic acid molecule or a portion thereof, and the sense region comprises a nucleotide sequence corresponding to the target nucleic acid sequence or a portion thereof.

Examples of chemical modifications that can be made in an siNA include phosphorothioate internucleotide linkages, 2′-deoxyribonucleotides, 2′-O-methyl ribonucleotides, 2′-deoxy-2′-fluoro ribonucleotides, “universal base” nucleotides, “acyclic” nucleotides, 5-C-methyl nucleotides, and terminal glyceryl and/or inverted deoxy abasic residue incorporation.

The antisense region of a siNA molecule can include a phosphorothioate internucleotide linkage at the 3′-end of said antisense region. The antisense region can comprise about one to about five phosphorothioate internucleotide linkages at the 5′-end of said antisense region. The 3′-terminal nucleotide overhangs of a siNA molecule can include ribonucleotides or deoxyribonucleotides that are chemically-modified at a nucleic acid sugar, base, or backbone. The 3′-terminal nucleotide overhangs can include one or more universal base ribonucleotides. The 3′-terminal nucleotide overhangs can comprise one or more acyclic nucleotides.

For example, a chemically-modified siNA can have 1, 2, 3, 4, 5, 6, 7, 8, or more phosphorothioate internucleotide linkages in one strand, or can have 1 to 8 or more phosphorothioate internucleotide linkages in each strand. The phosphorothioate internucleotide linkages can be present in one or both oligonucleotide strands of the siNA duplex, for example in the sense strand, the antisense strand, or both strands.

siNA molecules can comprise one or more phosphorothioate internucleotide linkages at the 3′-end, the 5′-end, or both of the 3′- and 5′-ends of the sense strand, the antisense strand, or in both strands. For example, an exemplary siNA molecule can include 1, 2, 3, 4, 5, or more consecutive phosphorothioate internucleotide linkages at the 5′-end of the sense strand, the antisense strand, or both strands.

In some embodiments, a siNA molecule includes 1, 2, 3, 4, 5, 6, 7, 8, 9, 10, or more pyrimidine phosphorothioate internucleotide linkages in the sense strand, the antisense strand, or in both strands.

In some embodiments, a siNA molecule includes 1, 2, 3, 4, 5, 6, 7, 8, 9, 10, or more purine phosphorothioate internucleotide linkages in the sense strand, the antisense strand, or in both strands.

A siNA molecule can include a circular nucleic acid molecule, wherein the siNA is about 38 to about 70, for example, about 38, 40, 45, 50, 55, 60, 65, or 70 nucleotides in length, having about 18 to about 23, for example, about 18, 19, 20, 21, 22, or 23 base pairs, 4wherein the circular oligonucleotide forms a dumbbell-shaped structure having about 19 base pairs and 2 loops.

A circular siNA molecule can contain two loop motifs, wherein one or both loop portions of the siNA molecule is biodegradable. For example, the loop portions of a circular siNA molecule may be transformed in vivo to generate a double-stranded siNA molecule with 3′-terminal overhangs, such as 3′-terminal nucleotide overhangs comprising about 2 nucleotides.

Modified nucleotides in a siNA molecule can be in the antisense strand, the sense strand, or both. For example, modified nucleotides can have a Northern conformation (e.g., Northern pseudorotation cycle, see, for example, Saenger, Principles of Nucleic Acid Structure, Springer-Verlag ed., 1984). Examples of nucleotides having a Northern configuration include locked nucleic acid (LNA) nucleotides (e.g., 2′-O, 4′-C-methylene-(D-ribofuranosyl) nucleotides), 2′-methoxyethoxy (MOE) nucleotides, 2′-methyl-thio-ethyl, 2′-deoxy-2′-fluoro nucleotides, 2′-deoxy-2′-chloro nucleotides, 2′-azido nucleotides, and 2′-O-methyl nucleotides.

Chemically modified nucleotides can be resistant to nuclease degradation while at the same time maintaining the capacity to mediate RNAi.

The sense strand of a double stranded siNA molecule may have a terminal cap moiety such as an inverted deoxyabasic moiety, at the 3′-end, 5′-end, or both 3′ and 5′-ends of the sense strand.

Examples of conjugates include conjugates and ligands described in Vargeese, et al., U.S. application Ser. No. 10/427,160, filed Apr. 30, 2003, incorporated by reference herein in its entirety, including the drawings.

In some embodiments of this invention, the conjugate may be covalently attached to the chemically-modified interfering RNA molecule via a biodegradable linker. For example, the conjugate molecule may be attached at the 3′-end of either the sense strand, the antisense strand, or both strands of the chemically-modified interfering RNA molecule.

In some embodiments, the conjugate molecule is attached at the 5′-end of either the sense strand, the antisense strand, or both strands of the chemically-modified siNA molecule. In some embodiments, the conjugate molecule is attached both the 3′-end and 5′-end of either the sense strand, the antisense strand, or both strands of the chemically-modified siNA molecule, or any combination thereof.

In some embodiments, a conjugate molecule comprises a molecule that facilitates delivery of a chemically-modified siNA molecule into a biological system, such as a cell.

In some embodiments, a conjugate molecule attached to the chemically-modified siNA molecule is a polyethylene glycol, human serum albumin, or a ligand for a cellular receptor that can mediate cellular uptake. Examples of specific conjugate molecules contemplated by the instant invention that can be attached to chemically-modified siNA molecules are described in Vargeese, et al., U.S. Patent Publication No. 20030130186 and U.S. Patent Publication No. 20040110296, which are each hereby incorporated by reference in their entirety.

A siNA may be contain a nucleotide, non-nucleotide, or mixed nucleotide/non-nucleotide linker that joins the sense region of the siNA to the antisense region of the siNA. In some embodiments, a nucleotide linker can be 3, 4, 5, 6, 7, 8, 9, or 10 nucleotides in length. In some embodiments, the nucleotide linker can be a nucleic acid aptamer. As used herein, the terms “aptamer” or “nucleic acid aptamer” encompass a nucleic acid molecule that binds specifically to a target molecule, wherein the nucleic acid molecule contains a sequence that is recognized by the target molecule in its natural setting. Alternately, an aptamer can be a nucleic acid molecule that binds to a target molecule where the target molecule does not naturally bind to a nucleic acid.

For example, the aptamer can be used to bind to a ligand-binding domain of a protein, thereby preventing interaction of the naturally occurring ligand with the protein. See, for example, Gold, et al., Annu. Rev. Biochem. 64:763, 1995; Brody and Gold, J. Biotechnol. 74:5, 2000; Sun, Curr. Opin. Mol. Ther. 2:100, 2000; Kusser, J. Biotechnol. 74:27, 2000; Hermann and Patel, Science 287:820, 2000; and Jayasena, Clinical Chemistry 45:1628, 1999.

A non-nucleotide linker can be an abasic nucleotide, polyether, polyamine, polyamide, peptide, carbohydrate, lipid, polyhydrocarbon, or other polymeric compounds (e.g., polyethylene glycols such as those having between 2 and 100 ethylene glycol units). Specific examples include those described by Seela and Kaiser, Nucleic Acids Res. 18:6353, 1990, and Nucleic Acids Res. 15:3113, 1987; Cload and Schepartz, J. Am. Chem. Soc. 113:6324, 1991; Richardson and Schepartz, J. Am. Chem. Soc. 113:5109, 1991; Ma, et al., Nucleic Acids Res. 21:2585, 1993, and Biochemistry 32:1751, 1993; Durand, et al., Nucleic Acids Res. 18:6353, 1990; McCurdy, et al, Nucleosides & Nucleotides 10:287, 1991; Jschke, et al., Tetrahedron Lett. 34:301, 1993; Ono, et al., Biochemistry 30:9914, 1991; Arnold, et al., International Publication No. WO 89/02439; Usman, et al., International Publication No. WO 95/06731; Dudycz, et al., International Publication No. WO 95/11910, and Ferentz and Verdine, J. Am Chem. Soc. 113:4000, 1991.

A “non-nucleotide linker” refers to a group or compound that can be incorporated into a nucleic acid chain in the place of one or more nucleotide units, including either sugar and/or phosphate substitutions, and allows the remaining bases to exhibit their enzymatic activity. The group or compound can be abasic in that it does not contain a commonly recognized nucleotide base, such as adenosine, guanine, cytosine, uracil or thymine, for example at the C1 position of the sugar.

In some embodiments, modified siNA molecule can have phosphate backbone modifications including one or more phosphorothioate, phosphorodithioate, methylphosphonate, phosphotriester, morpholino, amidate carbamate, carboxymethyl, acetamidate, polyamide, sulfonate, sulfonamide, sulfamate, formacetal, thioformacetal, and/or alkylsilyl substitutions. Examples of oligonucleotide backbone modifications are given in Hunziker and Leumann, Nucleic Acid Analogues: Synthesis and Properties, in Modern Synthetic Methods, VCH, pp. 331-417, 1995, and Mesmaeker, et al., Novel Backbone Replacements for Oligonucleotides, in Carbohydrate Modifications in Antisense Research, ACS, pp. 24-39, 1994.

siNA molecules, which can be chemically-modified, can be synthesized by: (a) synthesis of two complementary strands of the siNA molecule; and (b) annealing the two complementary strands together under conditions suitable to obtain a double-stranded siNA molecule. In some embodiments, synthesis of the complementary portions of the siNA molecule is by solid phase oligonucleotide synthesis, or by solid phase tandem oligonucleotide synthesis.

Oligonucleotides (e.g., certain modified oligonucleotides or portions of oligonucleotides lacking ribonucleotides) are synthesized using protocols known in the art, for example as described in Caruthers, et al, Methods in Enzymology 211:3-19, 1992; Thompson, et al., International PCT Publication No. WO 99/54459; Wincott, et al., Nucleic Acids Res. 23:2677-2684, 1995; Wincott, et al., Methods Mol. Bio. 74:59, 1997; Brennan, et al., Biotechnol Bioeng. 61:33-45, 1998; and Brennan, U.S. Pat. No. 6,001,31 1. Synthesis of RNA, including certain siNA molecules of the invention, follows general procedures as described, for example, in Usman, et al., J. Am. Chem. Soc. 109:7845, 1987; Scaringe, et al., Nucleic Acids Res. 18:5433, 1990; and Wincott, et al, Nucleic Acids Res. 23:2677-2684, 1995; Wincott, et al, Methods Mol. Bio. 74:59, 1997.

An “asymmetric hairpin” as used herein is a linear siNA molecule comprising an antisense region, a loop portion that can comprise nucleotides or non-nucleotides, and a sense region that comprises fewer nucleotides than the antisense region to the extent that the sense region has enough complementary nucleotides to base pair with the antisense region and form a duplex with loop.

An “asymmetric duplex” as used herein is a siNA molecule having two separate strands comprising a sense region and an antisense region, wherein the sense region comprises fewer nucleotides than the antisense region to the extent that the sense region has enough complementary nucleotides to base pair with the antisense region and form a duplex.

To “modulate gene expression” as used herein is to upregulate or downregulate expression of a target gene, which can include upregulation or downregulation of mRNA levels present in a cell, or of mRNA translation, or of synthesis of protein or protein subunits, encoded by the target gene.

The terms “inhibit,” “down-regulate,” or “reduce expression,” as used herein mean that the expression of the gene, or level of RNA molecules or equivalent RNA molecules encoding one or more proteins or protein subunits, or level or activity of one or more proteins or protein subunits encoded by a target gene, is reduced below that observed in the absence of the nucleic acid molecules (e.g., siNA) of the invention.

“Gene silencing” as used herein refers to partial or complete inhibition of gene expression in a cell and may also be referred to as “gene knockdown.” The extent of gene silencing may be determined by methods known in the art, some of which are summarized in International Publication No. WO 99/32619.

As used herein, the terms “ribonucleic acid” and “RNA” refer to a molecule containing at least one ribonucleotide residue. A ribonucleotide is a nucleotide with a hydroxyl group at the 2′ position of a beta-D-ribo-furanose moiety. These terms include double-stranded RNA, single-stranded RNA, isolated RNA such as partially purified RNA, essentially pure RNA, synthetic RNA, recombinantly produced RNA, as well as modified and altered RNA that differs from naturally occurring RNA by the addition, deletion, substitution, modification, and/or alteration of one or more nucleotides. Alterations of an RNA can include addition of non-nucleotide material, such as to the end(s) of a siNA or internally, for example at one or more nucleotides of an RNA.

Nucleotides in an RNA molecule include non-standard nucleotides, such as non-naturally occurring nucleotides or chemically synthesized nucleotides or deoxynucleotides. These altered RNAs can be referred to as analogs.

By “highly conserved sequence region” is meant, a nucleotide sequence of one or more regions in a target gene does not vary significantly from one generation to the other or from one biological system to the other.

By “sense region” is meant a nucleotide sequence of a siNA molecule having complementarity to an antisense region of the siNA molecule. In addition, the sense region of a siNA molecule can comprise a nucleic acid sequence having homology with a target nucleic acid sequence.

By “antisense region” is meant a nucleotide sequence of a siNA molecule having complementarity to a target nucleic acid sequence. In addition, the antisense region of a siNA molecule can include a nucleic acid sequence having complementarity to a sense region of the siNA molecule.

By “target nucleic acid” is meant any nucleic acid sequence whose expression or activity is to be modulated. A target nucleic acid can be DNA or RNA.

By “complementarity” is meant that a nucleic acid can form hydrogen bond(s) with another nucleic acid sequence either by traditional Watson-Crick or by other non-traditional modes of binding.

The term “biodegradable linker” as used herein, refers to a nucleic acid or non-nucleic acid linker molecule that is designed as a biodegradable linker to connect one molecule to another molecule, for example, a biologically active molecule to a siNA molecule or the sense and antisense strands of a siNA molecule. The biodegradable linker is designed such that its stability can be modulated for a particular purpose, such as delivery to a particular tissue or cell type. The stability of a nucleic acid-based biodegradable linker molecule can be variously modulated, for example, by combinations of ribonucleotides, deoxyribonucleotides, and chemically-modified nucleotides, such as 2′-O-methyl, 2′-fluoro, 2′-amino, 2′-O-amino, 2′-C-allyl, 2′-O-allyl, and other 2′-modified or base modified nucleotides. The biodegradable nucleic acid linker molecule can be a dimer, trimer, tetramer or longer nucleic acid molecule, for example, an oligonucleotide of about 2, 3, 4, 5, 6, 7, 8, 9, 10, 11, 12, 13, 14, 15, 16, 17, 18, 19, or 20 nucleotides in length, or can comprise a single nucleotide with a phosphorus-based linkage, for example, a phosphoramidate or phosphodiester linkage. The biodegradable nucleic acid linker molecule can also comprise nucleic acid backbone, nucleic acid sugar, or nucleic acid base modifications.

In connection with 2′-modified nucleotides as described herein, by “amino” is meant 2′-NH₂ or 2′-O—NH₂, which can be modified or unmodified. Such modified groups are described, for example, in Eckstein, et al., U.S. Pat. No. 5,672,695 and Matulic-Adamic, et al., U.S. Pat. No. 6,248,878.

Supplemental or complementary methods for delivery of nucleic acid molecules for use within then invention are described, for example, in Akhtar et al., Trends Cell Bio. 2:139, 1992; “Delivery Strategies for Antisense Oligonucleotide Therapeutics,” ed. Akhtar, 1995, Maurer et al., Mol. Membr. Biol. 16:129-140, 1999; Hofland and Huang, Handb. Exp. Pharmacol. 13 7:165-192, 1999; and Lee et al., ACS Symp. Ser. 752:184-192, 2000. Sullivan, et al., International PCT Publication No. WO 94/02595, further describes general methods for delivery of enzymatic nucleic acid molecules.

Nucleic acid molecules can be administered within formulations that include one or more additional components, such as a pharmaceutically acceptable carrier, diluent, excipient, adjuvant, emulsifier, buffer, stabilizer, or preservative.

As used herein, the term “carrier” means a pharmaceutically acceptable solid or liquid filler, diluent or encapsulating material. A water-containing liquid carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. Examples of ingredients of the above categories can be found in the U.S. Pharmacopeia National Formulary, 1990, pp. 1857-1859, as well as in Raymond C. Rowe, et al., Handbook of Pharmaceutical Excipients, 5th ed., 2006, and “Remington: The Science and Practice of Pharmacy,” 21st ed., 2006, editor David B. Troy.

Examples of preservatives include phenol, methyl paraben, paraben, m-cresol, thiomersal, benzylalkonium chloride, and mixtures thereof.

Examples of surfactants include oleic acid, sorbitan trioleate, polysorbates, lecithin, phosphotidylcholines, various long chain diglycerides and phospholipids, and mixtures thereof.

Examples of phospholipids include phosphatidylcholine, lecithin, phosphatidylglycerol, phosphatidylinositol, phosphatidylserine, and phosphatidylethanolamine, and mixtures thereof.

Examples of dispersants include ethylenediaminetetraacetic acid.

Examples of gases include nitrogen, helium, chlorofluorocarbons (CFCs), hydrofluorocarbons (HFCs), carbon dioxide, air, and mixtures thereof.

In certain embodiments, the siNA and/or the polypeptide can be encapsulated in liposomes, administered by iontophoresis, or incorporated into other vehicles, such as hydrogels, cyclodextrins, biodegradable nanocapsules, bioadhesive microspheres, or proteinaceous vectors (see e.g., O'Hare and Normand, International PCT Publication No. WO 00/53722). Alternatively, a nucleic acid composition can be locally delivered by direct injection or by use of an infusion pump. Direct injection of the nucleic acid molecules of the invention, whether subcutaneous, intramuscular, or intradermal, can take place using standard needle and syringe methodologies, or by needle-free technologies such as those described in Conry et al., Clin. Cancer Res. 5:2330-2337, 1999, and Barry et al., International PCT Publication No. WO 99/31262.

The compositions of this invention can be effectively employed as pharmaceutical agents. Pharmaceutical agents prevent, modulate the occurrence or severity of, or treat (alleviate one or more symptom(s) to a detectable or measurable extent) of a disease state or other adverse condition in a patient.

In some embodiments, this invention provides pharmaceutical compositions and methods featuring the presence or administration of one or more polynucleic acid(s), typically one or more siNAs, combined, complexed, or conjugated with a lipid, which may further be formulated with a pharmaceutically-acceptable carrier, such as a diluent, stabilizer, or buffer.

Typically, the siNA will target a gene that is expressed at an elevated level as a causal or contributing factor associated with the subject disease state or adverse condition. In this context, the siNA will effectively downregulate expression of the gene to levels that prevent, alleviate, or reduce the severity or recurrence of one or more associated disease symptoms. Alternatively, for various distinct disease models where expression of the target gene is not necessarily elevated as a consequence or sequel of disease or other adverse condition, down regulation of the target gene will nonetheless result in a therapeutic result by lowering gene expression (i.e., to reduce levels of a selected mRNA and/or protein product of the target gene). Alternatively, siNAs of the invention may be targeted to lower expression of one gene, which can result in upregulation of a “downstream” gene whose expression is negatively regulated by a product or activity of the target gene.

This siNAs of the present invention may be administered in any form, for example transdermally or by local injection (e.g., local injection at sites of psoriatic plaques to treat psoriasis, or into the joints of patients afflicted with psoriatic arthritis or RA). In more detailed embodiments, the invention provides formulations and methods to administer therapeutically effective amounts of siNAs directed against of a mRNA of TNF-α, which effectively down-regulate the TNF-α RNA and thereby reduce or prevent one or more TNF-α-associated inflammatory condition(s). Comparable methods and compositions are provided that target expression of one or more different genes associated with a selected disease condition in animal subjects, including any of a large number of genes whose expression is known to be aberrantly increased as a causal or contributing factor associated with the selected disease condition.

The compositions of the present invention may also be formulated and used as tablets, capsules or elixirs for oral administration, suppositories for rectal administration, sterile solutions, suspensions for injectable administration, and the other forms known in the art.

A pharmacological composition or formulation refers to a composition or formulation in a form suitable for administration, e.g., systemic administration, into a cell or patient, including for example a human. Suitable forms, in part, depend upon the use or the route of entry, for example oral, transdermal, transepithelial, or by injection. Such forms should not prevent the composition or formulation from reaching a target cell (i.e., a cell to which the negatively charged nucleic acid is desirable for delivery). For example, pharmacological compositions injected into the blood stream should be soluble. Other factors are known in the art, and include considerations such as toxicity.

By “systemic administration” is meant in vivo systemic absorption or accumulation of drugs in the blood stream followed by distribution throughout the entire body. Administration routes which lead to systemic absorption include, without limitation: intravenous, subcutaneous, intraperitoneal, inhalation, oral, intrapulmonary and intramuscular.

Examples of agents suitable for formulation with the nucleic acid molecules of this invention include: P-glycoprotein inhibitors (such as Pluronic P85), which can enhance entry of drugs into the CNS (Jolliet-Riant and Tillement, Fundam. Clin. Pharmacol. 13:16-26, 1999); biodegradable polymers, such as poly (DL-lactide-coglycolide) microspheres for sustained release delivery after intracerebral implantation (Emerich, D. F., et al., Cell Transplant 8:47-58, 1999, Alkermes, Inc., Cambridge, Mass.); and loaded nanoparticles, such as those made of polybutylcyanoacrylate, which can deliver drugs across the blood brain barrier and can alter neuronal uptake mechanisms (Prog. Neuropsychopharmacol Biol. Psychiatry 23:941-949, 1999). Other examples of delivery strategies for the nucleic acid molecules of the instant invention include material described in Boado, et al., J. Pharm. Sci. 87:1308-1315, 1998; Tyler, et al., FEBS Lett. 421:280-284, 1999; Pardridge, et al., PNAS USA. 92:5592-5596, 1995; Boado, Adv. Drug Delivey Rev. 15:73-107, 1995; Aldrian-Herrada et al., Nucleic Acids Res. 26:4910-4916, 1998; and Tyler, et al., PNAS USA. 96:7053-7058, 1999.

The present invention also includes compositions prepared for storage or administration, which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A.R. Gennaro ed. 1985). For example, preservatives, stabilizers, dyes and flavoring agents may be provided. These include sodium benzoate, sorbic acid and esters of p-hydroxybenzoic acid. In addition, antioxidants and suspending agents may be used.

A pharmaceutically effective dose is that dose required to prevent, inhibit the occurrence of, treat, or alleviate a symptom to some extent of a disease state. An amount of from 0.01 mg/kg to 50 mg/kg body weight/day of active nucleic acid should be administered.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono-or diglycerides. In addition, fatty acids such as oleic acid find use in the preparation of injectables.

The siNAs can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

The siNAs can be modified extensively to enhance stability by modification with nuclease resistant groups, for example, 2′-amino, 2′-C-allyl, 2′-fluoro, 2′-O-methyl, 2′-H. For a review see Usman and Cedergren, TIBS 1 7:34, 1992; Usman, et al., Nucleic Acids Symp. Ser. 31:163, 1994. SiNA constructs can be purified by gel electrophoresis using general methods or can be purified by high pressure liquid chromatography and re-suspended in water.

Chemically synthesizing nucleic acid molecules with modifications (base, sugar and/or phosphate) can prevent their degradation by serum ribonucleases, which can increase their potency. See e.g., Eckstein, et al., International Publication No. WO 92/07065; Perrault et al., Nature 344:565, 1990; Pieken, et al, Science 253, 314, 1991; Usman and Cedergren, Trends in Biochem. Sci. 17:334, 1992; Usman, et al., International Publication No. WO 93/15187; and Rossi et al., International Publication No. WO 91/03162; Sproat, U.S. Pat. No. 5,334,711; Gold, et al., U.S. Pat. No. 6,300,074. All of the above references describe various chemical modifications that can be made to the base, phosphate and/or sugar moieties of the nucleic acid molecules described herein.

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-fluoro, 2′-O-methyl, 2′-O-allyl, 2′-H, nucleotide base modifications. For a review see Usman and Cedergren, TIBS 17:34, 1992; Usman, et al, Nucleic Acids Symp. Ser. 31:163, 1994; Burgin, et al, Biochemistry 35:14090, 1996. Sugar modification of nucleic acid molecules have been extensively described in the art. See Eckstein et al., International Publication PCT No. WO 92/07065; Perrault, et al. Nature 344:565-568, 1990; Pieken, et al. Science 253:314-317, 1991; Usman and Cedergren, Trends in Biochem. Sci. 17:334-339, 1992; Usman et al. International Publication PCT No. WO 93/15187; Sproat, U.S. Pat. No. 5,334,711 and Beigelman, et al., J. Biol. Chem. 270:25702, 1995; Beigelman, et al., International PCT Publication No. WO 97/26270; Beigelman, et al., U.S. Pat. No. 5,716,824; Usman, et al., U.S. Pat. No. 5,627,053; Woolf, et al., International PCT Publication No. WO 98/13526; Thompson, et al., Karpeisky, et al, Tetrahedron Lett. 39:1131, 1998; Earnshaw and Gait, Biopolymers (Nucleic Acid Sciences) 48:39-55, 1998; Verma and Eckstein, Annu. Rev. Biochem. 67:99-134, 1998; and Burlina, et al., Bioorg. Med. Chem. 5:1999-2010, 1997. Such publications describe general methods and strategies to determine the location of incorporation of sugar, base and/or phosphate modifications and the like into nucleic acid molecules without modulating catalysis. In view of such teachings, similar modifications can be used as described herein to modify the siNA nucleic acid molecules of the instant invention so long as the ability of siNA to promote RNAi in cells is not significantly inhibited.

While chemical modification of oligonucleotide internucleotide linkages with phosphorothioate, phosphorodithioate, and/or 5′-methylphosphonate linkages improves stability, excessive modifications can cause some toxicity or decreased activity. Therefore, when designing nucleic acid molecules, the amount of these internucleotide linkages should be minimized. The reduction in the concentration of these linkages should lower toxicity, resulting in increased efficacy and higher specificity of these molecules.

In one embodiment, the invention features modified siNA molecules, with phosphate backbone modifications comprising one or more phosphorothioate, phosphorodithioate, methylphosphonate, phosphotriester, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications, see Hunziker and Leumann, Nucleic Acid Analogues: Synthesis and Properties, in Modern Synthetic Methods, VCH, 1995, pp. 331-417, and Mesmaeker, et al., “Novel Backbone Replacements for Oligonucleotides, in Carbohydrate Modifications in Antisense Research,” ACS, 1994, pp. 24-39.

Methods for the delivery of nucleic acid molecules are described in Akhtar, et al., Trends Cell Bio. 2:139, 1992; “Delivery Strategies for Antisense Oligonucleotide Therapeutics,” ed. Akhtar, 1995; Maurer, et al., Mol. Membr. Biol. 16:129-140, 1999; Hofland and Huang, Handb. Exp. Pharmacol. 137:165-192, 1999; and Lee, et al., ACS Symp. Ser. 752:184-192, 2000. Beigelman, et al., U.S. Pat. No. 6,395,713, and Sullivan et al., PCT WO 94/02595 further describe the general methods for delivery of nucleic acid molecules. These protocols can be utilized for the delivery of virtually any nucleic acid molecule. Nucleic acid molecules can be administered to cells by a variety of methods known to those of skill in the art, including, but not restricted to, encapsulation in liposomes, by iontophoresis, or by incorporation into other vehicles, such as biodegradable polymers, hydrogels, cyclodextrins (see for example, Gonzalez, et al., Bioconjugate Chem. 10:1068-1074, 1999; Wang, et al., International PCT Publication Nos. WO 03/47518 and WO 03/46185), poly(lactic-co-glycolic)ac-id (PLGA) and PLCA microspheres (see for example U.S. Pat. No. 6,447,796 and U.S. Patent Application Publication No. US 2002130430), biodegradable nanocapsules, and bioadhesive microspheres, or by proteinaceous vectors (O'Hare and Normand, International PCT Publication No. WO 00/53722). Alternatively, the nucleic acid/vehicle combination is locally delivered by direct injection or by use of an infusion pump. Direct injection of the nucleic acid molecules of the invention, whether subcutaneous, intramuscular, or intradermal, can take place using standard needle and syringe methodologies, or by needle-free technologies such as those described in Conry, et al., Clin. Cancer Res. 5:2330-2337, 1999, and Barry, et al., International PCT Publication No. WO 99/31262. The molecules of the instant invention can be used as pharmaceutical agents. Pharmaceutical agents prevent, modulate the occurrence, or treat (alleviate a symptom to some extent, preferably all of the symptoms) of a disease state in a subject.

By “RNA” is meant a molecule comprising at least one ribonucleotide residue. By “ribonucleotide” is meant a nucleotide with a hydroxyl group at the 2′ position of a .beta.-D-ribo-furanose moiety. The terms include double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siNA or internally, for example at one or more nucleotides of the RNA. Nucleotides in the RNA molecules of the instant invention can also comprise non-standard nucleotides, such as non-naturally occurring nucleotides or chemically synthesized nucleotides or deoxynucleotides. These altered RNAs can be referred to as analogs or analogs of naturally-occurring RNA.

By “cap structure” is meant chemical modifications, which have been incorporated at either terminus of the oligonucleotide (see, for example, Adamic, et al, U.S. Pat. No. 5,998,203, incorporated by reference herein). These terminal modifications protect the nucleic acid molecule from exonuclease degradation, and may help in delivery and/or localization within a cell. The cap may be present at the 5′-terminus (5′-cap) or at the 3′-terminal (3′-cap) or may be present on both termini. In non-limiting examples, the 5′-cap includes, but is not limited to, glyceryl, inverted deoxy abasic residue (moiety); 4′,5′-methylene nucleotide; 1-(beta-D-erythrofuranosyl) nucleotide, 4′-thio nucleotide; carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety.

Examples of the 3′-cap include, but are not limited to, glyceryl, inverted deoxy abasic residue (moiety), 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Lyer, Tetrahedron 49:1925, 1993; incorporated by reference herein).

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine and therefore lacks a base at the 1′-position.

By “nucleotide” as used herein is as recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see, for example, Usman and McSwiggen, supra; Eckstein, et al., International PCT Publication No. WO 92/07065; Usman, et al, International PCT Publication No. WO 93/15187; Uhlman & Peyman, supra, all are hereby incorporated by reference herein). There are several examples of modified nucleic acid bases known in the art as summarized by Limbach, et al, Nucleic Acids Res. 22:2183, 1994. Some of the non-limiting examples of base modifications that can be introduced into nucleic acid molecules include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and others (Burgin, et al., Biochemistry 35:14090, 1996; Uhlman & Peyman, supra). By “modified bases” in this aspect is meant nucleotide bases other than adenine, guanine, cytosine and uracil at 1′ position or their equivalents.

By “target site” or “target sequence” or “targeted sequence” is meant a sequence within a target nucleic acid (e.g., RNA) that is “targeted” for cleavage mediated by a siNA construct which contains sequences within its antisense region that are complementary to the target sequence.

The siNA molecules can be complexed with cationic lipids, packaged within liposomes, or otherwise delivered to target cells or tissues. The nucleic acid or nucleic acid complexes can be locally administered to through injection, infusion pump or stent, with or without their incorporation in biopolymers. In another embodiment, polyethylene glycol (PEG) can be covalently attached to siNA compounds of the present invention, to the polypeptide, or both. The attached PEG can be any molecular weight, preferably from about 2,000 to about 50,000 daltons (Da).

The sense region can be connected to the antisense region via a linker molecule, such as a polynucleotide linker or a non-nucleotide linker.

“Inverted repeat” refers to a nucleic acid sequence comprising a sense and an antisense element positioned so that they are able to form a double stranded siRNA when the repeat is transcribed. The inverted repeat may optionally include a linker or a heterologous sequence such as a self-cleaving ribozyme between the two elements of the repeat. The elements of the inverted repeat have a length sufficient to form a double stranded RNA. Typically, each element of the inverted repeat is about 15 to about 100 nucleotides in length, preferably about 20-30 base nucleotides, preferably about 20-25 nucleotides in length, e.g., 20, 21, 22, 23, 24, 25, 26, 27, 28, 29, or 30 nucleotides in length.

“Nucleic acid” refers to deoxyribonucleotides or ribonucleotides and polymers thereof in single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2′-O-methyl ribonucleotides, peptide-nucleic acids (PNAs).

“Large double-stranded RNA” refers to any double-stranded RNA having a size greater than about 40 bp for example, larger than 100 bp or more particularly larger than 300 bp. The sequence of a large dsRNA may represent a segment of a mRNA or the entire mRNA. The maximum size of the large dsRNA is not limited herein. The double-stranded RNA may include modified bases where the modification may be to the phosphate sugar backbone or to the nucleoside. Such modifications may include a nitrogen or sulfur heteroatom or any other modification known in the art.

The double-stranded structure may be formed by self-complementary RNA strand such as occurs for a hairpin or a micro RNA or by annealing of two distinct complementary RNA strands.

“Overlapping” refers to when two RNA fragments have sequences which overlap by a plurality of nucleotides on one strand, for example, where the plurality of nucleotides (nt) numbers as few as 2-5 nucleotides or by 5-10 nucleotides or more.

“One or more dsRNAs” refers to dsRNAs that differ from each other on the basis of primary sequence.

“Target gene or mRNA” refers to any gene or mRNA of interest. Indeed any of the genes previously identified by genetics or by sequencing may represent a target. Target genes or mRNA may include developmental genes and regulatory genes as well as metabolic or structural genes or genes encoding enzymes. The target gene may be expressed in those cells in which a phenotype is being investigated or in an organism in a manner that directly or indirectly impacts a phenotypic characteristic. The target gene may be endogenous or exogenous. Such cells include any cell in the body of an adult or embryonic animal or plant including gamete or any isolated cell such as occurs in an immortal cell line or primary cell culture.

All publications, references, patents, patent publications and patent applications cited herein are each hereby specifically incorporated by reference in its entirety.

While this invention has been described in relation to certain embodiments, and many details have been set forth for purposes of illustration, it will be apparent to those skilled in the art that this invention includes additional embodiments, and that some of the details described herein may be varied considerably without departing from this invention. This invention includes such additional embodiments, modifications and equivalents. In particular, this invention includes any combination of the features, terms, or elements of the various illustrative components and examples.

The use herein of the terms “a,” “an,” “the,” and similar terms in describing the invention, and in the claims, are to be construed to include both the singular and the plural. The terms “comprising,” “having,” “including,” and “containing” are to be construed as open-ended terms which mean, for example, “including, but not limited to.” Recitation of a range of values herein refers individually to each and any separate value falling within the range as if it were individually recited herein, whether or not some of the values within the range are expressly recited. Specific values employed herein will be understood as exemplary and not to limit the scope of the invention.

Definitions of technical terms provided herein should be construed to include without recitation those meanings associated with these terms known to those skilled in the art, and are not intended to limit the scope of the invention.

The examples given herein, and the exemplary language used herein are solely for the purpose of illustration, and are not intended to limit the scope of the invention.

When a list of examples is given, such as a list of compounds or molecules suitable for this invention, it will be apparent to those skilled in the art that mixtures of the listed compounds or molecules are also suitable.

### EXAMPLE 1

### In vitro Assay for LacZ Gene Knockdown in 9L Cells

9L/LacZ cells were transfected with various lipid formulations. An example of the protocol is shown below:
1. Plate 9L/LacZ cells at 4.0×10³ cells/well (96-well plate) and incubate overnight. Confluency about 15-20% at the time of transfection next day.
2. Add 0.5 μl dsRNA (20 μM stock) into 12 μl Opti-MEM, mixed by vortex.
3. Dilute lipid formulation (1 mM stock) into 12.5 μl Opti-MEM, vortex 10 sec, keep at room temperature for 5 min. Final concentration for HiPerFect™ (positive control): 7.5 μM.
4. Mix #2 and #3 together by vortex 10 sec. Keep at room temperature for 10-15 min. Total volume 25 μl (transfection complex).
5. Replace overnight cell culture media with 75 μl fresh complete media (DMEM plus 10% fetal bovine serum).
6. Add 25 μl transfection complex to each well. Total volume will be 100 μl. Incubate the cells at 37° C., 5% CO2 overnight.
7. Replace with 100 μl fresh media and score (scale 0-10) fluorescence intensity (uptake) under fluorescence microscope by visual inspection.
   β-Galactosidase Assay. 9L/LacZ cells are harvested at day 3 post transfection.
1. Wash 9L/LacZ cells (96-well plate) once with 100 μl phosphate buffered saline (PBS).
2. Add 50 μl lysis buffer (M-PER® Reagent, Pierce) to each well.
3. Incubate plate at room temperature for 15 minutes.
4. Transfer 10 μl lysate from each well to a new plate (96-well) for protein assay with Micro BCA kit (Pierce) (next section).
5. Transfer another 30 μl lysate from each well to another new plate (96-well) for β-galactosidase assay.
6. Add 30 μl All-in-One™ β-Galactosidase Assay Reagent (Pierce) to each well.
7. Cover plate and incubate for 30 minutes at 37° C.
8. Measure absorbance with 96-well plate reader at 405 nm.
   Micro BCA assay for quantifying protein concentration.
1. Transfer 10 μl lysate to each well in a 96-well new plate (described as above)
2. Add 140 μl water into each well, so total volume will be 150 μl.
3. Add 150 μl micro BCA working solution (25:24:1 of Reagent A:B:C, Pierce) to each well.
4. Cover plate and incubate for 2 hr at 37° C.
5. Measure absorbance at 562 nm.

### EXAMPLE 2

Example formulations of some compositions of this disclosure are shown in Table 6.

TABLE 6
Example Formulations

| | | Non- | | | | | |
|---|---|---|---|---|---|---|---|
| | | cationic | Chol | VitE | DHA | | |
| | DOTMA | lipid | Mol | Mol | Mol | siRNA | |
| No. | Mol % | Mol % | % | % | % | pmol | N/P |
| LC119 | 24 | 48 (DOPE) | 20 | 5 | 3 | 10 | 1.22 |
| LC128 | 24 | 48 (DPPC) | 20 | 5 | 3 | 10 | 1.22 |
| LC137 | 24 | 48 (DSPC) | 20 | 5 | 3 | 10 | 1.22 |

An approximately 50 to 1 molar ratio was provided in these formulations. 0.5 uL of 1 mM monocationic component provided 500 pmol total cation per transfection (amount N). 0.5 uL of a 20 uM stock siRNA provided 10 pmols dsRNA per transfection (amount P, x41 for a 41 nucleotide dsRNA duplex). DOTMA stock concentration was 1.0 nM. Charge Ratios were kept constant throughout all transfection experiments and were set at an N/P ratio of 50/41=1.22.

### EXAMPLE 3

Example formulations of compositions of this invention are shown in Table 7.

TABLE 7
Example Formulations

| No. | Composition |
|---|---|
| LC001 | DDPC/DOTAP(2:1) |
| LC002 | DDPC/DOTMA(2:1) |
| LC003 | DDPC/18:0 DDAB(2:1) |
| LC004 | DDPC/18:1 DAP(2:1) |
| LC005 | DDPC/14:0 TAP(2:1) |
| LC006 | DDPC/18:0 TAP(2:1) |
| LC007 | DDPC/18:1 EPC(2:1) |
| LC008 | DDPC/POEPC(2:1) |
| LC009 | DDPC/16:0 EPC(2:1) |
| LC010 | PLinPC/DOTAP(2:1) |
| LC011 | PLinPC/DOTMA(2:1) |
| LC012 | PLinPC/18:0 DDAB(2:1) |
| LC013 | PLinPC/18:1 DAP(2:1) |
| LC014 | PLinPC/14:0 TAP(2:1) |
| LC015 | PLinPC/18:0 TAP(2:1) |
| LC016 | PLinPC/18:1 EPC(2:1) |
| LC017 | PLinPC/POEPC(2:1) |
| LC018 | PLinPC/16:0 EPC(2:1) |
| LC019 | DLPC/DOTAP(2:1) |
| LC020 | DLPC/DOTMA(2:1) |
| LC021 | DLPC/18:0 DDAB(2:1) |
| LC022 | DLPC/18:1 DAP(2:1) |
| LC023 | DLPC/14:0 TAP(2:1) |
| LC024 | DLPC/18:0 TAP(2:1) |
| LC025 | DLPC/18:1 EPC(2:1) |
| LC026 | DLPC/POEPC(2:1) |
| LC027 | DLPC/16:0 EPC(2:1) |
| LC028 | DMPC/DOTAP(2:1) |
| LC029 | DMPC/DOTMA(2:1) |
| LC030 | DMPC/18:0 DDAB(2:1) |
| LC031 | DMPC/18:1 DAP(2:1) |
| LC032 | DMPC/14:0 TAP(2:1) |
| LC033 | DMPC/18:0 TAP(2:1) |
| LC034 | DMPC/18:1 EPC(2:1) |
| LC035 | DMPC/POEPC(2:1) |
| LC036 | DMPC/16:0 EPC(2:1) |
| LC037 | DOPE/DOTAP(2:1) |
| LC038 | DOPE/DOTMA(2:1) |
| LC039 | DOPE/18:0 DDAB(2:1) |
| LC040 | DOPE/18:1 DAP(2:1) |
| LC041 | DOPE/14:0 TAP(2:1) |
| LC042 | DOPE/18:0 TAP(2:1) |
| LC043 | DOPE/18:1 EPC(2:1) |
| LC044 | DOPE/POEPC(2:1) |
| LC045 | DOPE/16:0 EPC(2:1) |
| LC046 | DPPC/DOTAP(2:1) |
| LC047 | DPPC/DOTMA(2:1) |
| LC048 | DPPC/18:0 DDAB(2:1) |
| LC049 | DPPC/18:1 DAP(2:1) |
| LC050 | DPPC/14:0 TAP(2:1) |
| LC051 | DPPC/18:0 TAP(2:1) |
| LC052 | DPPC/18:1 EPC(2:1) |
| LC053 | DPPC/POEPC(2:1) |
| LC054 | DPPC/16:0 EPC(2:1) |
| LC055 | DSPC/DOTAP(2:1) |
| LC056 | DSPC/DOTMA(2:1) |
| LC057 | DSPC/18:0 DDAB(2:1) |
| LC058 | DSPC/18:1 DAP(2:1) |
| LC059 | DSPC/14:0 TAP(2:1) |
| LC060 | DSPC/18:0 TAP(2:1) |
| LC061 | DSPC/18:1 EPC(2:1) |
| LC062 | DSPC/POEPC(2:1) |
| LC063 | DSPC/16:0 EPC(2:1) |
| LC064 | POPC/DOTAP(2:1) |
| LC065 | POPC/DOTMA(2:1) |
| LC066 | POPC/18:0 DDAB(2:1) |
| LC067 | POPC/18:1 DAP(2:1) |
| LC068 | POPC/14:0 TAP(2:1) |
| LC069 | POPC/18:0 TAP(2:1) |
| LC070 | POPC/18:1 EPC(2:1) |
| LC071 | POPC/POEPC(2:1) |
| LC072 | POPC/16:0 EPC(2:1) |
| LC073 | DPhPC/DOTAP(2:1) |
| LC074 | DPhPC/DOTMA(2:1) |
| LC075 | DPhPC/18:0 DDAB(2:1) |
| LC076 | DPhPC/18:1 DAP(2:1) |
| LC077 | DPhPC/14:0 TAP(2:1) |
| LC078 | DPhPC/18:0 TAP(2:1) |
| LC079 | DPhPC/18:1 EPC(2:1) |
| LC080 | DPhPC/POEPC(2:1) |
| LC081 | DPhPC/16:0 EPC(2:1) |
| LC082 | DDPC/DOTAP(2:1) + Chol/VitE/DHA |
| LC083 | DDPC/DOTMA(2:1) + Chol/VitE/DHA |
| LC084 | DDPC/18:0 DDAB(2:1) + Chol/VitE/DHA |
| LC085 | DDPC/18:1 DAP(2:1) + Chol/VitE/DHA |
| LC086 | DDPC/14:0 TAP(2:1) + Chol/VitE/DHA |
| LC087 | DDPC/18:0 TAP(2:1) + Chol/VitE/DHA |
| LC088 | DDPC/18:1 EPC(2:1) + Chol/VitE/DHA |
| LC089 | DDPC/POEPC(2:1) + Chol/VitE/DHA |
| LC090 | DDPC/16:0 EPC(2:1) + Chol/VitE/DHA |
| LC091 | PLinPC/DOTAP(2:1) + Chol/VitE/DHA |
| LC092 | PLinPC/DOTMA(2:1) + Chol/VitE/DHA |
| LC093 | PLinPC/18:0 DDAB(2:1) + Chol/VitE/DHA |
| LC094 | PLinPC/18:1 DAP(2:1) + Chol/VitE/DHA |
| LC095 | PLinPC/14:0 TAP(2:1) + Chol/VitE/DHA |
| LC096 | PLinPC/18:0 TAP(2:1) + Chol/VitE/DHA |
| LC097 | PLinPC/18:1 EPC(2:1) + Chol/VitE/DHA |
| LC098 | PLinPC/POEPC(2:1) + Chol/VitE/DHA |
| LC099 | PLinPC/16:0 EPC(2:1) + Chol/VitE/DHA |
| LC100 | DLPC/DOTAP(2:1) + Chol/VitE/DHA |
| LC101 | DLPC/DOTMA(2:1) + Chol/VitE/DHA |
| LC102 | DLPC/18:0 DDAB(2:1) + Chol/VitE/DHA |
| LC103 | DLPC/18:1 DAP(2:1) + Chol/VitE/DHA |
| LC104 | DLPC/14:0 TAP(2:1) + Chol/VitE/DHA |
| LC105 | DLPC/18:0 TAP(2:1) + Chol/VitE/DHA |
| LC106 | DLPC/18:1 EPC(2:1) + Chol/VitE/DHA |
| LC107 | DLPC/POEPC(2:1) + Chol/VitE/DHA |
| LC108 | DLPC/16:0 EPC(2:1) + Chol/VitE/DHA |
| LC109 | DMPC/DOTAP(2:1) + Chol/VitE/DHA |
| LC110 | DMPC/DOTMA(2:1) + Chol/VitE/DHA |
| LC111 | DMPC/18:0 DDAB(2:1) + Chol/VitE/DHA |
| LC112 | DMPC/18:1 DAP(2:1) + Chol/VitE/DHA |
| LC113 | DMPC/14:0 TAP(2:1) + Chol/VitE/DHA |
| LC114 | DMPC/18:0 TAP(2:1) + Chol/VitE/DHA |
| LC115 | DMPC/18:1 EPC(2:1) + Chol/VitE/DHA |
| LC116 | DMPC/POEPC(2:1) + Chol/VitE/DHA |
| LC117 | DMPC/16:0 EPC(2:1) + Chol/VitE/DHA |
| LC118 | DOPE/DOTAP(2:1) + Chol/VitE/DHA |
| LC119 | DOPE/DOTMA(2:1) + Chol/VitE/DHA |
| LC120 | DOPE/18:0 DDAB(2:1) + Chol/VitE/DHA |
| LC121 | DOPE/18:1 DAP(2:1) + Chol/VitE/DHA |
| LC122 | DOPE/14:0 TAP(2:1) + Chol/VitE/DHA |
| LC123 | DOPE/18:0 TAP(2:1) + Chol/VitE/DHA |
| LC124 | DOPE/18:1 EPC(2:1) + Chol/VitE/DHA |
| LC125 | DOPE/POEPC(2:1) + Chol/VitE/DHA |
| LC126 | DOPE/16:0 EPC(2:1) + Chol/VitE/DHA |
| LC127 | DPPC/DOTAP(2:1) + Chol/VitE/DHA |
| LC128 | DPPC/DOTMA(2:1) + Chol/VitE/DHA |
| LC129 | DPPC/18:0 DDAB(2:1) + Chol/VitE/DHA |
| LC130 | DPPC/18:1 DAP(2:1) + Chol/VitE/DHA |
| LC131 | DPPC/14:0 TAP(2:1) + Chol/VitE/DHA |
| LC132 | DPPC/18:0 TAP(2:1) + Chol/VitE/DHA |
| LC133 | DPPC/18:1 EPC(2:1) + Chol/VitE/DHA |
| LC134 | DPPC/POEPC(2:1) + Chol/VitE/DHA |
| LC135 | DPPC/16:0 EPC(2:1) + Chol/VitE/DHA |
| LC136 | DSPC/DOTAP(2:1) + Chol/VitE/DHA |
| LC137 | DSPC/DOTMA(2:1) + Chol/VitE/DHA |
| LC138 | DSPC/18:0 DDAB(2:1) + Chol/VitE/DHA |
| LC139 | DSPC/18:1 DAP(2:1) + Chol/VitE/DHA |
| LC140 | DSPC/14:0 TAP(2:1) + Chol/VitE/DHA |
| LC141 | DSPC/18:0 TAP(2:1) + Chol/VitE/DHA |
| LC142 | DSPC/18:1 EPC(2:1) + Chol/VitE/DHA |
| LC143 | DSPC/POEPC(2:1) + Chol/VitE/DHA |
| LC144 | DSPC/16:0 EPC(2:1) + Chol/VitE/DHA |
| LC145 | POPC/DOTAP(2:1) + Chol/VitE/DHA |
| LC146 | POPC/DOTMA(2:1) + Chol/VitE/DHA |
| LC147 | POPC/18:0 DDAB(2:1) + Chol/VitE/DHA |
| LC148 | POPC/18:1 DAP(2:1) + Chol/VitE/DHA |
| LC149 | POPC/14:0 TAP(2:1) + Chol/VitE/DHA |
| LC150 | POPC/18:0 TAP(2:1) + Chol/VitE/DHA |
| LC151 | POPC/18:1 EPC(2:1) + Chol/VitE/DHA |
| LC152 | POPC/POEPC(2:1) + Chol/VitE/DHA |
| LC153 | POPC/16:0 EPC(2:1) + Chol/VitE/DHA |
| LC154 | DPhPC/DOTAP(2:1) + Chol/VitE/DHA |
| LC155 | DPhPC/DOTMA(2:1) + Chol/VitE/DHA |
| LC156 | DPhPC/18:0 DDAB(2:1) + Chol/VitE/DHA |
| LC157 | DPhPC/18:1 DAP(2:1) + Chol/VitE/DHA |
| LC158 | DPhPC/14:0 TAP(2:1) + Chol/VitE/DHA |
| LC159 | DPhPC/18:0 TAP(2:1) + Chol/VitE/DHA |
| LC160 | DPhPC/18:1 EPC(2:1) + Chol/VitE/DHA |
| LC161 | DPhPC/POEPC(2:1) + Chol/VitE/DHA |
| LC162 | DPhPC/16:0 EPC(2:1) + Chol/VitE/DHA |

### EXAMPLE 4

Knockdown in vitro assays of lac-z expression in rat gliosarcoma fibroblast cells 9L/LacZ for the example formulations of Table 7 are shown in Table 8. Comparative data using HiPerFect™ (Qiagen; Valencia, Calif.) is also shown.

TABLE 8
LacZ Knockdown for siRNA Lipid Compositions

| | LacZ |
|---|---|
| Formulation ID | Expression |
| Untransfected | 1.00 |
| HiPerFect | 0.18 |
| 1 | 0.7 |
| 2 | 1.0 |
| 3 | 0.3 |
| 4 | 1.1 |
| 5 | 0.7 |
| 6 | 0.7 |
| 7 | 0.9 |
| 8 | 1.0 |
| 9 | 0.9 |
| 10 | 1.0 |
| 11 | 0.8 |
| 12 | 0.9 |
| 13 | 0.9 |
| 14 | 0.8 |
| 15 | 1.0 |
| 16 | 0.9 |
| 17 | 0.9 |
| 18 | 1.0 |
| 19 | 0.7 |
| 20 | 0.8 |
| 21 | 0.7 |
| 22 | 1.2 |
| 23 | 0.8 |
| 24 | 1.0 |
| 25 | 1.1 |
| 26 | 1.1 |
| 27 | 1.1 |
| 28 | 0.8 |
| 29 | 1.0 |
| 30 | 0.5 |
| 31 | 1.2 |
| 32 | 1.0 |
| 33 | 0.9 |
| 34 | 1.0 |
| 35 | 1.0 |
| 36 | 0.9 |
| 37 | 0.2 |
| 38 | 0.2 |
| 39 | 0.1 |
| 40 | 0.9 |
| 41 | 0.3 |
| 42 | 0.6 |
| 43 | 0.7 |
| 44 | 0.6 |
| 45 | 0.8 |
| 46 | 1.0 |
| 47 | 0.8 |
| 48 | 0.8 |
| 49 | 1.2 |
| 50 | 0.9 |
| 51 | 0.8 |
| 52 | 1.1 |
| 53 | 0.9 |
| 54 | 1.2 |
| 55 | 0.7 |
| 56 | 0.8 |
| 57 | 0.8 |
| 58 | 1.3 |
| 59 | 0.8 |
| 60 | 1.0 |
| 61 | 1.1 |
| 62 | 0.9 |
| 63 | 1.0 |
| 64 | 1.4 |
| 65 | 1.1 |
| 66 | 0.9 |
| 67 | 0.9 |
| 68 | 0.4 |
| 69 | 0.8 |
| 70 | 1.1 |
| 71 | 1.1 |
| 72 | 1.4 |
| 73 | 0.9 |
| 74 | 0.7 |
| 75 | 0.7 |
| 76 | 1.7 |
| 77 | 0.9 |
| 78 | 1.7 |
| 79 | 1.1 |
| 80 | 1.2 |
| 81 | 0.7 |
| 82 | 0.8 |
| 83 | 0.9 |
| 84 | 1.3 |
| 85 | 0.9 |
| 86 | 0.6 |
| 87 | 0.6 |
| 88 | 1.0 |
| 89 | 0.9 |
| 90 | 0.8 |
| 91 | 1.0 |
| 92 | 0.9 |
| 93 | 0.8 |
| 94 | 0.9 |
| 95 | 0.6 |
| 96 | 0.9 |
| 97 | 1.1 |
| 98 | 1.1 |
| 99 | 0.9 |
| 100 | 0.8 |
| 101 | 0.9 |
| 102 | 0.6 |
| 103 | 1.2 |
| 104 | 0.4 |
| 105 | 0.6 |
| 106 | 1.0 |
| 107 | 0.8 |
| 108 | 0.9 |
| 109 | 0.8 |
| 110 | 0.8 |
| 111 | 0.8 |
| 112 | 1.1 |
| 113 | 0.8 |
| 114 | 0.8 |
| 115 | 1.1 |
| 116 | 1.0 |
| 117 | 1.2 |
| 118 | 0.2 |
| 119 | 0.3 |
| 120 | 0.1 |
| 121 | 1.0 |
| 122 | 0.2 |
| 123 | 0.3 |
| 124 | 0.5 |
| 125 | 0.6 |
| 126 | 0.6 |
| 127 | 0.5 |
| 128 | 0.4 |
| 129 | 0.5 |
| 130 | 0.9 |
| 131 | 0.5 |
| 132 | 1.0 |
| 133 | 1.0 |
| 134 | 1.1 |
| 135 | 1.2 |
| 136 | 0.8 |
| 137 | 0.3 |
| 138 | 0.8 |
| 139 | 1.2 |
| 140 | 0.7 |
| 141 | 0.8 |
| 142 | 0.9 |
| 143 | 0.9 |
| 144 | 1.0 |
| 145 | 1.0 |
| 146 | 1.0 |
| 147 | 0.7 |
| 148 | 1.3 |
| 149 | 0.8 |
| 150 | 0.8 |
| 151 | 0.9 |
| 152 | 0.9 |
| 153 | 0.6 |
| 154 | 0.9 |
| 155 | 0.7 |
| 156 | 0.8 |
| 157 | 1.1 |
| 158 | 0.7 |
| 159 | 1.1 |
| 160 | 0.7 |
| 161 | 0.9 |
| 162 | 0.6 |

What is claimed is:

1. A pharmaceutical composition comprising a double-stranded RNA (dsRNA), one or more cationic lipids, one or more non-cationic lipids, and one or more lipophilic delivery-enhancing compounds.

2. The composition of claim 1, wherein the lipophilic delivery-enhancing compound is an omega-3 fatty acid.

3. The composition of claim 1, wherein the lipophilic delivery-enhancing compound is selected from octadeca-9,12,15-trienoic acid, octadeca-6,9,12,15-tetraenoic acid, eicosa-8,11,14,17-tetraenoic acid, eicosa-5,8,11,14,17-pentaenoic acid, docosa-7,10,13,16,19-pentaenoic acid, docosa-4,7,10,13,16,19-hexaenoic acid, and mixtures thereof.

4. The composition of claim 1, wherein the lipophilic delivery-enhancing compound is eicosa-5,8,11,14,17-pentaenoic acid or docosa-4,7,10,13,16,19-hexaenoic acid.

5. The composition of claim 1, wherein the lipophilic delivery-enhancing compound is docosa-4,7,10,13,16,19-hexaenoic acid.

6. The composition of claim 1, wherein the lipophilic delivery-enhancing compound is selected from Vitamin E, tocol (2-methyl-2-(4,8,12-trimethyltridecyl)chroman-6-ol), tocotrienols, tocopherols, α-tocopherol, 2-methyl-2-(4,8,12-trimethyltrideca-3,7,11-trienyl)chroman-6-ol (E,E)-tocotrienol), and mixtures thereof.

7. The composition of claim 1, wherein the lipophilic delivery-enhancing compound is Vitamin E or α-tocopherol.

8. The composition of claim 1, wherein the lipophilic delivery-enhancing compound is Vitamin E.

9. The composition of claim 1, wherein the cationic lipid is selected from the group consisting of DOTMA, DOTAP, DMTAP, DMRIE, DDAB, DC-chol, BGTC, RPR209120, 1,2-dioleoyl-sn-glycero-3-ethylphosphocholine, 1,2-distearoyl-sn-glycero-3-ethylphosphocholine, 1,2-dipalmitoyl-sn-glycero-3-ethylphosphocholine, TMTPS, TMTOS, TMTLS, TMTMS, TMDOS, DOGS, DOGS-9-en, DLinGS, GL-67, DOSPER, DOSPA, and mixtures thereof.

10. The composition of claim 1, wherein the cationic lipid is selected from DOTMA, DOTAP, DDAB, 14:0 TAP, 18:0 TAP, and mixtures thereof.

11. The composition of claim 1, wherein the cationic lipid is DOTMA.

12. The composition of claim 1, wherein the non-cationic lipid is selected from cholesterol, DDPE. DLPE, DSPE, DOPE, DLinPE, DLenPE, DARAPE, DDHAPE, DPhPE, DSPC, DPPC, DDPC, DPPS, DSPS, and mixtures thereof.

13. The composition of claim 1, wherein the non-cationic lipid is selected from DOPE, DPPC, DSPC, and mixtures thereof.

14. The composition of claim 1, wherein the non-cationic lipid is DOPE.

15. The composition of claim 1, wherein the dsRNA is an siRNA or an shRNA.

16. The composition of claim 1, wherein the dsRNA has a 3′ overhang.

17. The composition of claim 1, wherein the dsRNA has a 3′ overhang containing a deoxythymidine (dT).

18. The composition of claim 1, wherein the dsRNA is G1498.

19. The composition of claim 1, wherein the dsRNA is an shRNA further comprising a hairpin loop structure.

20. The composition of claim 1, wherein the dsRNA is an shRNA further comprising a hairpin loop structure having from 4 to 11 nucleotides.

21. The composition of claim 1, wherein the composition decreases expression of an influenza A virus gene by at least about 25% in a mammalian cell.

22. The composition of claim 1, comprising 24 mole percent DOTMA, 48 mole percent DOPE, 20 mole percent cholesterol, 5 mole percent vitamin E, and 3 mole percent DHA.

23. A method for delivering a dsRNA to a cell comprising preparing a composition according to claim 1 and treating the cell with the composition.

24. A method for inhibiting expression of a gene in a mammal comprising preparing a composition according to claim 1 and administering the composition to the mammal.

25. A method for treating influenza in a mammal comprising preparing a composition according to claim 1 and administering an effective amount of the composition to the mammal.
